(19) United States
(12) Patent Application Publication
(10) Pub. No.: US 2005/0214285 A1
(43) Pub. Date: Sep. 29, 2005

(54) Epitopes or mimotopes derived from the C-epsilon-3 or C-epsilon-4 domains of IgE, antagonists thereof, and their therapeutic uses

(75) Inventors: Michael Dyson, Cambidge (GB); Martin Friede, Cardiff, CA (US); Judith Greenwood, Cambridge (GB); Ellen Hewitt, Royston (GB); Alan Lamont, Croydon (GB); Sean Mason, Cambridge (GB); Roger Randall, Colne (GB); William Gordon Turnell, Cambridge (GB); Marcelle Paulette Van Mechelen, Wagnelee (BE); Carlotta Vinals y de Bassols, Brussels (BE)

Correspondence Address: GLAXOSMITHKLINE;Corporate Intellectual Property - UW2220, P.O. Box 1539, King of Prussia, PA, 19406-0939, US

(73) Assignee: SmithKline Beecham Biologicals, s.a. and Peptide Therapeutics Limited

(21) Appl. No.: 11/005,794

(22) Filed: Dec. 7, 2004

Related U.S. Application Data
(63) Continuation of application No. 09/914,088, filed on Nov. 13, 2001, now abandoned.

(30) Foreign Application Priority Data
Mar. 29, 1999 (GB) 9907151.6
May 7, 1999 (GB) 9910537.1
May 7, 1999 (GB) 9910538.9
Aug. 7, 1999 (GB) 9918594.4
Aug. 7, 1999 (GB) 9918603.3
Sep. 7, 1999 (GB) 9921046.0
Sep. 7, 1999 (GB) 9921047.8
Oct. 29, 1999 (GB) 9925619.0
Nov. 23, 1999 (GB) 9927698.2

(51) Int. Cl. G01N 33/53; A61K 39/395; C07K 16/42

(52) U.S. Cl. 424/144.1; 530/388.22

(57) ABSTRACT

The present invention relates to the provision of novel medicaments for the treatment, prevention or amelioration of allergic disease. In particular, the novel medicaments are isolated peptides incorporating epitopes or mimotopes of surface exposed regions of the Cε2 domain of IgE. The inventors have found that these novel regions may be the target for both passive and active immunoprophylaxis or immunotherapy. The invention further relates to methods for production of the medicaments, pharmaceutical compositions containing them and their use in medicine. Also forming an aspect of the present invention are ligands, especially monoclonal antibodies, which are capable of binding the surface exposed IgE regions of the present invention, and their use in medicine as passive immunotherapy or in immunoprophylaxis.

The present invention relates to the provision of novel medicaments for the treatment, prevention or amelioration of allergic disease. In particular, the novel medicaments are isolated peptides incorporating epitopes or mimotopes of surface exposed regions of the Cε2 domain of IgE. The inventors have found that these novel regions may be the target for both passive and active immunoprophylaxis or immunotherapy. The invention further relates to methods for production of the medicaments, pharmaceutical compositions containing them and their use in medicine. Also forming an aspect of the present invention are ligands, especially monoclonal antibodies, which are capable of binding the surface exposed IgE regions of the present invention, and their use in medicine as passive immunotherapy or in immunoprophylaxis. Non-peptidic mimotopes are also an embodiment of the present invention.

In an allergic response, the symptoms commonly associated with allergy are brought about by the release of allergic mediators, such as histamine, from immune cells into the surrounding tissues and vascular structures. Histamine is normally stored in mast cells and basophils, until such time as the release is triggered by interaction with allergen specific IgE. The role of IgE in the mediation of allergic responses, such as asthma, food allergies, atopic dermatitis, type-I hypersensitivity and allergic rhinitis, is well known. On encountering an antigen, such as pollen or dust mite allergens, B-cells commence the synthesis of allergen specific IgE. The allergen specific IgE then binds to the FcεRI receptor (the high affinity IgE receptor) on basophils and mast cells. Any subsequent encounter with allergen leads to the triggering of histamine release from the mast cells or basophils, by cross-linking of neighbouring IgE/ FcεRI complexes (Sutton and Gould, Nature, 1993, 366: 421-428; EP 0477231 B1).

IgE, like all immunoglobulins, comprises two heavy and two light chains. The c heavy chain consists of five domains: one variable domain (VH) and four constant domains (Cε1 to Cε4). The molecular weight of IgE is about 190,000 Da, the heavy chain being approximately 550 amino acids in length. The structure of IgE is discussed in Padlan and Davis (Mol. Immunol., 23, 1063-75, 1986) and Helm et al., (2IgE model structure deposited Feb. 10, 1990 with PDB (Protein Data Bank, Research Collabarotory for Structural Bioinformatics; http:pdb-browsers.ebi.ac.uk)). The second domain, Cε2, approximately comprises amino acids 226-328 of IgE (Flanagan J. G. and Rabbitts, T. H., 1982, EMBO J., 1, 655-660; Kenten et al., 1982, Proc. Natl. Acad. Sci., USA, 79, 6661-6665), but may encompass additional amino acids. By comparison with the known structure of IgG1, the start point of Cε3 domain is deduced to be Ser337.

In the past, a number of passive or active immunotherapeutic approaches designed to interfere with IgE-mediated histamine release mechanism have been lo investigated with variable success. These approaches include interfering with IgE or allergen/IgE complexes binding to the FCεRI or FcεRII (the low affinity IgE receptor) receptors, with either passively administered antibodies, or with passive administration of IgE derived peptides to competitively bind to the receptors. In addition, some authors have described the use of specific peptides derived from IgE in active immunisation to stimulate histamine release-inhibiting immune responses.

It has been reported that the IgE domains involved in the binding of IgE to its receptor are Cε3 and Cε4 (Sutton, B. J. and Gould, H. J.; Nature, 1993, 366: 421-428; WO 97/31948), and as such the previous therapeutic strategies have focussed on portions of these two domains.

In the course of their investigations, previous workers in this field have encountered a number of considerations, and problems, which have to be taken into account when designing new anti-allergy therapies. One of the most dangerous problems revolves around the involvement of IgE cross-linking in the histamine release signal. It is most often the case that anti-IgE antibodies generated during active vaccination, are capable of triggering histamine release per se, by the cross-linking of neighbouring IgE-receptor complexes in the absence of allergen. This phenomenon is termed anaphylactogenicity. Indeed many commercially available anti-IgE monoclonal antibodies which are normally used for IgE detection assays, are anaphylactogenic, and consequently useless and potentially dangerous if administered to a patient.

Whether or not an antibody is anaphylactogenic depends on the location of the target epitope on the IgE molecule. However, based on the present state of knowledge in this area, and despite enormous scientific interest and endeavour, there is little or no predictability of what characteristics any antibody or epitope may have and whether or not it might have a positive or negative clinical effect on a patient.

Therefore, in order to be safe and effective, the passively administered, or vaccine induced, antibodies must bind in a region of IgE which is capable of interfering with the histamine triggering pathway, without being anaphylactic per se. The present invention achieves all of these aims and provides medicaments which are capable of raising non-anaphylactic antibodies which inhibit histamine release. These medicaments can form the basis of an active vaccine or be used to raise appropriate antibodies for passive immunotherapy, or may be passively administered themselves for a therapeutic effect.

Much work has been carried out by those skilled in the art to identify specific anti-IgE antibodies which do have some beneficial effects against IgE-mediated allergic reaction (WO 90/15878, WO 89/04834, WO 93/05810). Attempts have also been made to identify epitopes recognised by these useful antibodies, to create peptide mimotopes of such epitopes and to use those as immunogens to produce anti-IgE antibodies.

WO 97/31948 describes an example of this type of work, and further describes IgE peptides from the Cε3 and Cε4 domains conjugated to carrier molecules for active vaccination purposes. These immunogens may be used in vaccination studies and are said to be capable of generating antibodies which subsequently inhibit histamine release in vivo. In this work, a monoclonal antibody (BSW17) was described which was said to be capable of binding to IgE peptides contained within the Cε3 domain which are useful for active vaccination purposes.

EP 0 477 231 B1 describes immunogens derived from the Cε4 domain of IgE (residues 497-506, also known as the Stanworth decapeptide), conjugated to Keyhole Limpet Haemocyanin (KLH) used in active vaccination immunoprophylaxis. WO 96/14333 is a continuation of the work described in EP 0 477 231 B1.

Other approaches are based on the identification of peptides which themselves compete for IgE binding to the high or low affinity receptors on basophils or mast cells (WO 93/04173, WO 98/24808, EP 0 303 625 B1, EP 0 341 290).

The present invention identifies novel surface exposed epitopes of the Cε2 domain of IgE, which may be used as the target of active or passive immuno-prophylaxis or therapy of allergic disease states. The present invention provides peptides incorporating the isolated epitopes per se, and further provides mimotopes of these newly identified epitopes, which may be used per se in the treatment of allergy, or may be used in immunogens in active vaccination immunoprophylaxis or therapy. The isolated epitopes or mimotopes of the present invention are preferably used in immunogens for active vaccination protocols to induce auto anti-IgE antibodies, which themselves limit, reduce, or eliminate allergic responses or symptoms in vaccinated subjects. Alternatively, the mimotopes or the immunogens of the present invention may be passively administered to a patient to limit, reduce, or eliminate allergic responses or symptoms in vaccinated subjects.

The peptides, which incorporate the isolated epitopes of the present invention are immunogenic, when suitably presented (e.g. on a carrier), and are capable of inducing auto anti-IgE antibodies which are non-anaphylactogenic, and function in ameliorating allergic responses in vivo. The epitopes or mimotopes of the present invention are preferably exclusively derived from Cε2 domain, in that they are not derived from any other domain, ie. they are not found within the Cε1, Cε3 or Cε4 domains. In particular, as a preferred embodiment they are derived from the domain encoded by Ser222-Ala329 of human IgE.

Specific epitopes of the Cε2 domain which have been found to be particularly suitable for use in the mimotopes or immunogens of the present invention are those which have been found by the present inventors to be surface exposed. The surface exposure of a region of IgE may be determined from its modelled structure. (Padlan and Davies, Mol. Immunol., 23, 1063-75, 1986; Helm et al., 2IgE model structure deposited Feb. 10, 1990 with PDB (Protein Data Banke, Research Collabarotory for Structural Bioinformatics)). The present inventors have found that the epitopes useful in the present invention, are also found to be highly surface exposed. From this observation the present inventors have designed a method for providing other suitable epitopes, those being epitopes having highly accessible regions calculated over a sliding window of five residues. The inventors have found that preferred regions of the Cε2 domain have an accessible surface calculated over a sliding window of 5 residues using the Molecular Simulations Software (MSI) of greater than 50 Å², and preferably greater than 80 Å².

Examples of such surface exposed Cε2 IgE epitopes are:

| Peptide | | Location sequence and | SEQ |
|---|---|---|---|
| Name | Sequence | IgE Domain | ID NO. |
| P1 | EDGQVMDVD | Cε2 (Glu270-Asp278) | 1 |
| P2 | STTQEGEL | Cε2 (Ser283-Leu290) | 2 |
| P3 | SQKHWLSDRT | Cε2 (Ser300-Thr309) | 3 |
| P4 | GHTFEDSTKK | Cε2 (Gly318-Lys327) | 4 |
| P5 | GGGHFPPT | Cε2 (Gly245-Thr250) | 5 |
| P6 | PGTINI | Cε2 (Pro262-Ile267) | 6 |
| P7 | FTPPT | Cε2 (Phe231-Thr235) | 7 |

Peptides incorporating such epitopes form a preferred aspect of the present invention. Mimotopes which have the same characteristics as these epitopes, and immunogens comprising such mimotopes which generate an immune response which cross-react with the IgE Cε2 epitope in the context of the IgE molecule, also form part of the present invention.

The present invention, therefore, includes the isolated peptides encompassing the native IgE epitopes themselves, and any mimotope thereof. The meaning of mimotope is defined as an entity which is sufficiently similar to the native IgE epitope so as to be capable of being recognised by antibodies which recognise the native IgE epitope; (Gheysen, H. M., et al., 1986, Synthetic peptides as antigens. Wiley, Chichester, Ciba foundation symposium 119, p130-149; Gheysen, H. M., 1986, Molecular Immunology, 23,7, 709-715); or are capable of raising antibodies, when coupled to a suitable carrier, which antibodies cross-react with the native IgE epitope.

The mimotopes of the present invention may be peptidic or non-peptidic. A peptidic mimotope of the surface exposed IgE epitopes identified above, may have a sequence which differs from the native epitope but may also be of exactly the same sequence as the native epitope. Such a molecule is described as a mimotope of the epitope, because although the two molecules share the same sequence, the mimotope will not be presented in the context of the whole Cε2 domain structure, and as such the mimotope may take a slightly different conformation to that of the native IgE epitope. It will also be clear to the man skilled in the art that the above identified linear sequences (P1 to P7), when in the tertiary structure of IgE, lie adjacent to other regions that may be distant in the primary sequence of IgE. As such, for example, a mimotope of P1 may be continuous or discontinuous, in that it comprises or mimics segments of P1 and segments made up of these distant amino acid residues.

Preferred surface exposed regions which may be used in the present invention contain regions which are associated with a loop structure. The peptides or mimotopes of the present invention may comprise, therefore, a loop with N or C terminal extensions which may be the natural amino acid residues from neighbouring β-sheets. As examples of this P1 contains the C-D loop, P2 contains the D-E loop, P3 contains the E-F loop, P4 contains the F-G loop, P5 contains the A-B loop, and P6 contains the B-C loop of the Cε2 domain of IgE. Accordingly, mimotopes of these loops form an aspect of the present invention.

Particularly preferred medicaments are based on the epitope P1, and mimotopes thereof. Peptides incorporating this epitope, and mimotopes thereof, when coupled to a carrier, are potent in inducing anti-IgE immune responses which are capable of inhibiting histamine release from human basophils. Moreover, these immune responses are non-anaphylactogenic. Mimotopes of P1 are described primarily as any entity which when formulated into an immunogen, is capable of inducing an immune response, which response is capable of recognising P1 when in the context of Cε2 domain of IgE.

P1 corresponds to the C-D loop of the Cε2 domain. The C-D loop structure of immunoglobulin folds corresponds to the linking chain between the end of the C beta-strand and the beginning of the D beta-strand (Introduction to protein Structure, page 304, 2^{nd} Edition, Branden and Tooze, Garland Publishing, New York, ISBN 0 8153 2305-0), corresponding approximately to amino acid residue numbers Trp268-Ser280 of the IgE molecule. Accordingly, mimotopes of the C-D loop of IgE Cε2, and ligands that are capable of binding to the C-D loop of [gE Cε2, form a preferred aspect of the present invention.

Peptide mimotopes of the above-identified IgE epitopes may be designed for a particular purpose by addition, deletion or substitution of elected amino acids. Thus, the peptides of the present invention may be modified for the purposes of ease of conjugation to a protein carrier. For example, it may be desirable for some chemical conjugation methods to include a terminal cysteine to the IgE epitope. In addition it may be desirable for peptides conjugated to a protein carrier to include a hydrophobic terminus distal from the conjugated terminus of the peptide, such that the free unconjugated end of the peptide remains associated with the surface of the carrier protein. This reduces the conformational degrees of freedom of the peptide, and thus increases the probability that the peptide is presented in a conformation which most closely resembles that of the IgE peptide as found in the context of the whole IgE molecule. For example, the peptides may be altered to have an N-terminal cysteine and a C-terminal hydrophobic amidated tail. Alternatively, the addition or substitution of a D-stereoisomer form of one or more of the amino acids may be performed to create a beneficial derivative, for example to enhance stability of the peptide. Those skilled in the art will realise that such modified peptides, or mimotopes, could be a wholly or partly non-peptide mimotope wherein the constituent residues are not necessarily confined to the 20 naturally occurring amino acids. In addition, these may be cyclised by techniques known in the art to constrain the peptide into a conformation that closely resembles its shape when the peptide sequence is in the context of the whole IgE molecule.

Examples of preferred cyclised peptides which contain a pair of cysteine residues to allow the formation of a disulphide bridge are PT1079 (SEQ ID NO. 14), PT1079GS (SEQ ID NO.15), PT1078 (SEQ ID NO.16), and P15q (SEQ ID NO. 11).

Further, those skilled in the art will realise that mimotopes or immunogens of the present invention may be longer than-the isolated epitopes, and may comprise the sequences disclosed herein. Accordingly, the mimotopes of the present invention may consist of addition of N and/or C terminal extensions of a number of other natural residues at one or both ends. The peptide mimotopes may also be retro sequences of the natural IgE sequences, in that the sequence orientation is reversed; or alternatively the sequences may be entirely or at least in part comprised of D-stereo isomer amino acids (inverso sequences). Also, the peptide sequences may be retro-inverso in character, in that the sequence orientation is reversed and the amino acids are of the D-stereoisomer form. Such retro or retro-inverso peptides have the advantage of being non-self, and as such may overcome problems of self-tolerance in the immune system (for example P15r—see below).

Alternatively, peptide mimotopes may be identified using antibodies which are capable themselves of binding to the IgE epitopes of the present invention using techniques such as phage display technology (EP 0 552 267 B1). This technique, generates a large number of peptide sequences which mimic the structure of the native peptides and are, therefore, capable of binding to anti-native peptide antibodies, but may not necessarily themselves share significant sequence homology to the native IgE peptide. This approach may have significant advantages by allowing the possibility of identifying a peptide with enhanced immunogenic properties (such as higher affinity binding characteristics to the IgE receptors or anti-IgE antibodies, or being capable of inducing polyclonal immune response which binds to IgE with higher affinity), or may overcome any potential self-antigen tolerance problems which may be associated with the use of the native peptide sequence. Additionally this technique allows the identification of a recognition pattern for each native-peptide in terms of its shared chemical properties amongst recognised mimotope sequences.

Preferred examples of modified peptide mimotopes and examples of bacteriophage derived mimotopes include:

| | | | |
|---|---|---|---|
| | | | SEQ |
| Peptide | Sequence | Description ID NO. | |

| | | | |
|---|---|---|---|
| P15 | CLEDGQVMDVDLL-NH₂ | P1 mimotope | 8 |
| P15r | LLDVDMVQGDELC-NH₂ | P1 retro | 9 |
| | | mimotope | |
| P15p | WLEDGQVMDVDLC | P1 mimotope | 10 |
| P15q | CLEDGQVMDVDLC | P1 mimotope | 11 |
| C67/8 | CFINKQMADLELCPRE | P1 mimotope | 12 |
| C67 | CFMNKQLADLELCPRE | P1 mimotope | 13 |
| PT1079 | CLEDGQVMDVDLCPREAAEGDK | P1 mimotope | 14 |
| PT1079GS | CLEDGQVMDVDLCGGSSGGP | P1 mimotope | 15 |
| PT1078 | CLEDGQVMDVDCPREAAEGDK | P1 mimotope | 16 |
| P14s | QVMDVDL | P1 mimotope | 17 |
| EEC39-I | KCREVWLGESETIMDCE | P1 mimotope | 18 |
| EEC39-J | ACREVWLGESETIMDCD | P1 mimotope | 19 |
| EEC39-10 | SCREVWLGESETVMDCG | P1 mimotope | 20 |
| EEC40-9 | NCQDLMLREDAGCWSKM | P1 mimotope | 21 |
| EEC47-3 | DCEEPMCSPVLLQQLKL | P1 mimotope | 22 |
| P15t | LEDGQVMDVD | P1 mimotope | 23 |
| P16 | CSTTQEGELA-NH₂ | P2 mimotope | 24 |
| P2sh | TTQEGE | P2 mimotope | 25 |
| P17 | CSQKHWLSDRT-NH₂ | P3 mimotope | 26 |
| P4ex | TYQGHTFEDSTKKCADSNPRGV | P4 mimotope | 27 |
| P5sh | GGHFPP | P5 mimotope | 28 |
| P5long1 | CSSCDGGGHFPPTIQC | P5 mimotope | 192 |
| P5long2 | CLQSSCDGGGHFPPTIQLLC | P5 mimotope | 193 |

In other mimotopes, the amino acid residues of P1, P2, P3, P4, P4, P5, P6 or P7 can each individually be replaced by the amino acid that most closely resembles that amino acid. For example, A may be substituted by V, L or I, as described in the following table.

| Original | Exemplary | Preferred |
|---|---|---|
| residue | substitutions | substitution |
| A | V, L, I | V |
| R | K, Q, N | K |
| N | Q, H, K, R | Q |
| D | E | E |
| C | S | S |
| Q | N | N |
| E | D | D |
| G | P, A | A |
| H | N, Q, K, R | R |
| I | L, V, M, A, F | L |
| L | I, V, M, A, F | I |
| K | R, Q, N | R |
| M | L, F, I | L |
| F | L, V, I, A, Y | L |
| P | A | A |
| S | T | T |
| T | S | S |
| W | Y, F | Y |
| Y | W, F, T, S | F |
| V | I, L, M, F, A | L |

Ligands which are capable of binding to, the surface exposed Cε2 IgE epitopes, and pharmaceutical compositions comprising them, form part of the present invention. Such ligands are capable of being used in passive prophylaxis or therapy, by administration of the ligands into a patient, for the amelioration of allergic disease. Examples of such useful ligands include monoclonal or polyclonal antibodies. For example, antibodies induced in one animal may be purified and passively administered to another animal for the prophylaxis or therapy of allergy. The peptides of the present invention may also be used for the generation of monoclonal antibody hybridomas (using known techniques e.g. Kohler and Milstein, Nature, 1975, 256, p495), humanised monoclonal antibodies or CDR grafted monoclonals, by techniques known in the art. Accordingly, in a related aspect of the present invention are ligands capable of binding to the surface exposed epitopes of the Cε2 domain of IgE. Example of such ligands are antibodies (or Fab fragments). Such antibodies may be used in passive immunoprophylaxis or immunotherapy, or may be used themselves in the identification of IgE peptide mimotopes.

The term “antibody” herein is used to refer to a molecule having a useful antigen binding specificity. Those skilled in the art will readily appreciate that this term may also cover polypeptides which are fragments of or derivatives of antibodies yet which can show the same or a closely similar functionality. Such antibody fragments or derivatives are intended to be encompassed by the term antibody as used herein.

Preferred ligands are monoclonal antibodies. Particularly preferred ligands are ligands of P1, and are preferably monoclonal antibodies. For example, PTmAb0011 is the reference name for a mouse IgG1-type monoclonal antibody deposited as Budapest Treaty patent deposit at ECACC (European Collection of Cell Cultures, Vaccine Research and Production Laboratory, Public Health Laboratory Service, Centre for Applied Microbiology Research, Porton Down, Salisbury, Wiltshire, SP4 OJG, UK) on Mar. 8^{th}, 1999 under Accession No. 99030805.

For example, PTmAb0011 recognises the C-D loop of Cε2, and is itself capable of recognising IgE when bound to its high affinity receptor on human basophils without causing degranulation, moreover it is able to block the passive sensitisation of non-allergic basophils by preventing the binding of IgE to FcεR1α, and inhibits LolP1-triggered histamine release in allergic basophils. Another monoclonal antibody which recognises the C-D loop of CF2 is PTmAb0005 (available from Sigma Chemicals Catalogue number 16510, clone number GE-1). The present invention provides this monoclonal antibody in a pharmaceutical composition.

The ligands of P1 have been used in bacteriophage panning techniques to identify new P1 mimotopes. For example a monoclonal antibody which is capable of recognising P1, bound bacteriophages expressing the following sequences:

| | |
|---|---|
| SEQ ID | Sequence |

| | | | | | | | | | | | | | | | | |
|---|---|---|---|---|---|---|---|---|---|---|---|---|---|---|---|---|
| 29 | C | F | I | N | K | Q | M | A | D | L | E | L | C | | | |
| 30 | C | F | M | N | K | Q | L | A | D | L | E | L | C | | | |
| 31 | K | C | R | E | V | W | L | G | E | S | E | T | I | M | D | C |

Other peptide mimotopes of the C-D loop of Cε2 IgE have been identified by bacteriophage panning with PTmAb0011 and PTmAb0005. Examples of such mimotopes include:

| | |
|---|---|
| Peptide P1 mimotope | SEQ NO. |

| | |
|---|---|
| (PTmAb0011 phage panning) | |
| HCQQVFFPQDYLWCQRG | SEQ ID No.32 |
| SCREVWLGGSEMIMDCE | SEQ ID No.33 |
| ECNQNLSGSLRHVDLNC | SEQ ID No.34 |
| DCEEPMCSPVLLQKLKP | SEQ ID No.35 |
| SCREVWLGGSEMIMDCE | SEQ ID No.36 |
| RCDQQLPRDSYTFCMMS | SEQ ID No.37 |
| SCPAFPREGDLCAPPTV | SEQ ID No.38 |
| FCPEPICSPPLSRMTLS | SEQ ID No.39 |
| VCDECVSRELAL | SEQ ID No.40 |
| WCLEPECAPGLL | SEQ ID No.41 |
| VCDECVSRELAL | SEQ ID No.42 |
| DCLSKGQMADLC | SEQ ID No.43 |
| SCQGREVRRECW | SEQ ID No.44 |
| WCREVWLGESETIMDCE | SEQ ID No.45 |
| ACREVWLGESETIMDCD - | SEQ ID No.46 |
| GCAEPKCWQALHQKLKP - | SEQ ID No.47 |
| (PTmAb0005 phage panning) | |
| ECRGPNMQMQDHCPTTD | SEQ ID No.48 |
| QCNAVLEGLQMVDHCWN | SEQ ID No.49 |
| CCVADPETQMTPSSEMF | SEQ ID No.50 |
| HCKNEFKKGQWTYSCSD | SEQ ID No.51 |
| QCRQFVMNQSEKEFGQC | SEQ ID No.52 |
| NCFMNKQLADLELCPRE | SEQ ID No.53 |
| SCAYTAQRQCSDVPNPG | SEQ ID No.54 |
| GCFMNKQMADLELCPRTAA | SEQ ID No.55 |
| ACFMNKQMADLELCPRVAA | SEQ ID No.56 |
| GCFINKQLADLELCPRVAA | SEQ ID No.57 |
| GCFMNKQLADWELCPRAAA | SEQ ID No.58 |
| ECFMNKQLADSELCPRVAA | SEQ ID No.59 |
| GCFMNKQLADPELCPREAE | SEQ ID No.60 |
| GCFMNKQLVDLELCPRGAA | SEQ ID No.61 |
| GCFMNKQLADLELCPREAA | SEQ ID No.62 |
| GCFMNKQQADLELCPRGAA | SEQ ID No.63 |
| GCFINKQMADLELCPREAA | SEQ ID No.64 |

Therefore, mimotopes of IgE Cε2 that are capable of binding to PTmAb0005 or PTmAb0011, and immunogens comprising these mimotopes, form an important aspect of the present invention. Vaccines comprising mimotopes that are capable of binding to PTmAb0005 or PTmAb0011 are useful in the treatment of allergy.

Without limiting the broader definition of P1 mimotopes, from these and other phage-sequences, a core pattern has been identified for a sub-set of a P1-like peptide. This pattern is a sub-set of P1 mimotopes, and describes its mimotopes in terms of the chemical properties of the amino acids in each position which are desirable for recognition to that particular anti-P1 monoclonal antibody:
y h x d h h a n a n x y
   wherein:
y . . . y Can be cyclised.
h Hydrophobic (cys;pro;gly;ala;val;ile;leu;trp;met;phe).
d Ionic bond donating (arg;lys;his;gln;asn;trp;tyr;thr;ser).
a Acidic (asp;glu).
n Ionically neutral/ non-polar (all except asp,glu,lys,arg).
x Any amino acid (n=0−3).

Accordingly, in one embodiment, mimotopes of P1 may be described by the general core feature y h x d h h a n a n x y. described above. The peptide P1 or mimotope thereof may be optionally flanked by other amino acids at either end to aid conjugation or for any other purpose.

A particularly preferred mimotopes of P1 is P15s (SEQ ID NO. 17), whose Q, M, and first D residues have been shown to be critical for PTmAb0011 and PTmAb0005 binding activity (see examples). Hence a mimotope formula for P15s, in which the non-essential residues were replaced by similar amino acids (as outlined above) would be:
Q, Xi, M, D, X₁, X₂, X₃
wherein X₁ is selected from V, I, L, M, F or A; X₂ is selected from D or E; and X₃ is selected from L, I, V, M, A or F.

Also forming an important aspect of the present invention is the use of PTmAb0005 and PTmAb0011 in the identification of novel mimotopes of IgE, for subsequent use in allergy therapy. As PTmAb0005 is commercially available, this ligand does not form a composition of the present invention, however, pharmaceutical compositions comprising PTmAb0005, and its use in the identification of P1 mimotopes, form two important aspects of the present invention.

Mimotopes of P2, P3, P4 and P5 also form an important aspect of the present invention. For example P16 and P17, are mimotopes of P2 and P3 respectively. These peptides, when suitably presented on carriers, are both capable of inducing strong anti-IgE antibody responses which are non anaphylactogenic.

In a preferred embodiment, the peptides incorporating the above identified epitopes or peptidic or non-peptidic mimotopes of the present invention will be of a small size, such that they mimic a region selected from the whole Cε2 domain. It is envisaged that peptidic mimotopes, therefore, should be less than 100 amino acids in length, preferably shorter than 75 amino acids, more preferably less than 50 amino acids, and most preferable within the range of 4 to 25 amino acids long. Specific examples of preferred peptide mimotopes are PT1079 and P15q, which are respectively 21 and 13 amino acids long. Non-peptidic mimotopes are envisaged to be of a similar size, in terms of molecular volume, to their peptidic counterparts.

It will be apparent to the man skilled in the art that techniques may be used to confirm the status of a specific construct as a mimotope. Such techniques include the following: The putative mimotope can be assayed to ascertain the immunogenicity of the construct, in that antisera raised by the putative mimotope cross-react with the native IgE molecule, and are also functional in blocking allergic mediator release from allergic effector cells. The specificity of these responses can be confirmed by competition experiments by blocking the activity of the antiserum with the mimotope itself or the native IgE, and/or specific monoclonal antibodies that are known to bind the surface exposed epitope within Cε2 of IgE. Specific examples of such monoclonal antibodies for use in the competition assays include, for example, PTmAb0005 and PTmAb0011, which would confirm the status of the putative mimotope as a mimotope of the C-D loop of the Cε2 domain of IgE.

In one embodiment of the present invention at least one peptide as hereinbefore described, incorporating an IgE epitope or mimotope, is linked to carrier molecules to form immunogens for vaccination protocols. Preferably the carrier molecules are not related to the native IgE molecule. The peptides or mimotopes may be linked via chemical covalent conjugation or by expression of genetically engineered fusion partners, optionally via a linker sequence.

The covalent coupling of the peptide to the immunogenic carrier can be carried out in a manner well known in the art. Thus, for example, for direct covalent coupling it is possible to utilise a carbodiimide, glutaraldehyde or (N-[γ-maleimidobutyryloxy]) succinimide ester, utilising common commercially available heterobifunctional linkers such as CDAP and SPDP (using manufacturers instructions). After the coupling reaction, the immunogen can easily be isolated and purified by means of a dialysis method, a gel filtration method, a fractionation method etc.

The types of carriers used in the immunogens of the present invention will be readily known to the man skilled in the art. The function of the carrier is to provide cytokine help in order to help induce an immune response against the IgE peptide. A non-exhaustive list of carriers which may be used in the present invention include: Keyhole limpet Haemocyanin (KLH), serum albumins such as bovine serum albumin (BSA), inactivated bacterial toxins such as tetanus or diptheria toxins (TT and DT), or recombinant fragments thereof (for example, Domain 1 of Fragment C of TT, or the translocation domain of DT), or the purified protein derivative of tuberculin (PPD). Alternatively the mimotopes or epitopes may be directly conjugated to liposome carriers, which may additionally comprise immunogens capable of providing T-cell help. Preferably the ratio of peptides to carrier is in the order of 1:1 to 20:1, and preferably each carrier should carry between 3-15 peptides.

In an embodiment of the invention a preferred carrier is Protein D from Haemophilus influenzae UP 0 594 610 B1). Protein D is an IgD-binding protein from Haemophilus influenzae and has been patented by Forsgren (WO 91/18926, granted EP 0 594 610 B1). In some circumstances, for example in recombinant immunogen expression systems it may be desirable to use fragments of protein D, for example Protein D1/3^{rd} (comprising the N-terminal 100-110 amino acids of protein D (GB 9717953.5)).

Another preferred method of presenting the IgE peptides of the present invention is in the context of a recombinant fusion molecule. For example, EP 0 421 635 B describes the use of chimeric hepadnavirus core antigen particles to present foreign peptide sequences in a virus-like particle. As such, immunogens of the present invention may comprise IgE peptides presented in chimeric particles consisting of hepatitis B core antigen. Additionally, the recombinant fusion proteins may comprise the mimotopes of the present invention and a carrier protein, such as NS1 of the influenza virus. For any recombinantly expressed protein which forms part of the present invention, the nucleic acid which encodes said immunogen also forms an aspect of the present invention.

Peptides used in the present invention can be readily synthesised by solid phase procedures well known in the art. Suitable syntheses may be performed by utilising “T-boc” or “F-moc” procedures. Cyclic peptides can be synthesised by the solid phase procedure employing the well-known “F-moc” procedure and polyamide resin in the fully automated apparatus. Alternatively, those skilled in the art will know the necessary laboratory procedures to perform the process manually. Techniques and procedures for solid phase synthesis are described in ‘Solid Phase Peptide Synthesis: A Practical Approach’ by E. Atherton and R. C. Sheppard, published by IRL at Oxford University Press (1989). Alternatively, the peptides may be produced by recombinant methods, including expressing nucleic acid molecules encoding the mimotopes in a bacterial or mammalian cell line, followed by purification of the expressed mimotope. Techniques for recombinant expression of peptides and proteins are known in the art, and are described in Maniatis, T., Fritsch, E. F. and Sambrook et al., Molecular cloning, a laboratory manual, 2nd Ed.; Cold Spring Harbor Laboratory Press, Cold Spring Harbor, N.Y. (1989).

The immunogens of the present invention may comprise the peptides as previously described, including mimotopes, or may be immunologically cross-reactive derivatives or fragments thereof. Also forming part of the present invention are portions of nucleic acid which encode the immunogens of the present invention or peptides, mimotopes or derivatives thereof In addition, the immunogens of the present invention may comprise more than one type of epitope, i.e. P1 and P2, in the same immunogen, or the mimotope itself may comprise more than one type of epitope.

The present invention, therefore, provides the use of novel peptides encompassing the epitopes or mimotopes of the present invention (as defined above), in the manufacture of pharmaceutical compositions for the prophylaxis or therapy of allergies. Immunogens comprising the mimotopes or peptides of the present invention, and carrier molecules are also provided for use in vaccines for the immunoprophylaxis or therapy of allergies. Accordingly, the mimotopes, peptides or immunogens of the present invention are provided for use in medicine, and in the medical treatment or prophylaxis of allergic disease. Accordingly, there is provided a method of treatment of allergy comprising the administration to a patient suffering from or susceptible to allergy, of a vaccine or medicament of the present invention.

Vaccines of the present invention, may advantageously also include an adjuvant. Suitable adjuvants for vaccines of the present invention comprise those adjuvants that are capable of enhancing the antibody responses against the IgE peptide immunogen. Adjuvants are well known in the art (Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X). Preferred adjuvants for use with immunogens of the present invention include aluminium or calcium salts (for example hydroxide or phosphate salts). Other adjuvants include saponin adjuvants such as QS21 (U.S. Pat. No. 5,057,540) and 3D-MPL (GB 2220 211).

The vaccines of the present invention will be generally administered for both priming and boosting doses. It is expected that the boosting doses will be adequately spaced, or preferably given yearly or at such times where the levels of circulating antibody fall below a desired level. Boosting doses may consist of the peptide in the absence of the original carrier molecule. Such booster constructs may comprise an alternative carrier or may be in the absence of any carrier.

In a further aspect of the present invention there is provided a vaccine as herein described for use in medicine.

The vaccine preparation of the present invention may be used to protect or treat a mammal susceptible to, or suffering from allergies, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. A preferred route of administration is via the transdermal route, for example by skin patches.

The amount of protein in each vaccine dose is selected as an amount which induces an immunoprotective response without significant, adverse side effects in typical vaccinees. Such amount will vary depending upon which specific immunogen is employed and how it is presented. Generally, it is expected that each dose will s comprise 1-1000 μg of protein, preferably 1-500 μg, preferably 1-100 μg, of which 1 to 50 μg is the most preferable range. An optimal amount for a particular vaccine can be ascertained by standard studies involving observation of appropriate immune responses in subjects. Following an initial vaccination, subjects may receive one or several booster immunisations adequately spaced.

Pharmaceutical compositions comprising the ligands, described above, also form an aspect of the present invention. Also provided are the use of the ligands in medicine, and in the manufacture of medicaments for the treatment of allergies.

Aspects of the present invention may also be used in diagnostic assays. For example, panels of ligands which recognise the different peptides of the present invention may be used in assaying titres of anti-IgE present in serum taken from patients. Moreover, the peptides may themselves be used to type the circulating anti-IgE. It may in some circumstances be appropriate to assay circulating anti-IgE levels, for example in atopic patients, and as such the peptides and poly/mono-clonal antibodies of the present invention may be used in the diagnosis of atopy. In addition, the peptides may be used to affinity remove circulating anti-IgE from the blood of patients before re-infusion of the blood back into the patient.

Also forming part of the present invention is a method of identifying peptide immunogens for the immunoprophylaxis or therapy of allergy comprising using a computer model of the structure of IgE, and identifying those peptides of the IgE which are surface exposed. These regions may then be formulated into immunogens and used in medicine. Accordingly, the use of PTmAb0005 and PTmAb0011 in the identification of peptides for use in allergy immunoprophylaxis or therapy forms part of the present invention.

Vaccine preparation is generally described in New Trends and Developments in Vaccines, edited by Voller et al., University Park Press, Baltimore, Md., U.S.A. 1978. Conjugation of proteins to macromolecules is disclosed by Likhite, U.S. Pat. No. 4,372,945 and by Armor et al., U.S. Pat. No. 4, 474,757.

The numbering system for IgE amino acid residues is often that described by Dorington K J and Bennich H (1978) Immunol Rev 41 3-25; and also Bennich H and Bahr-Lindastrom, H von (1978) Prog Immunol 11 49-58. However, subsequent determination of the gene and cDNA sequence of human IgE (Max, E. E. et al 1982, Cell 29 691-699; Flanagan J. G. and Rabbitts, T. H., 1982, supra; Kenten, J. H. et al 1982, supra) revealed an extra leucine at position 273 (Kabat numbering) in Cε2 which was not reported in the earlier papers. The numbering scheme used by the present inventors may, therefore, differ from that used by Dorrington K J and Bennich.

### DESCRIPTION OF DRAWINGS

FIG. 1, IgE amino acid surface exposure using the Padlan and Davies 1986 model.

FIG. 2, Chemistry Scheme 1, solid phase peptide synthesis.

FIG. 3, Chemistry Scheme 2 and Scheme 3, Modified carrier preparation.

FIG. 4, Chemistry Scheme 4, Peptide/carrier conjugation.

FIG. 5, C67-8 Anti-IgE Data. (A) Anti-plate bound IgE reactivity of serum from Balb C mice immunised with 25 kg BSA-IgE C67-8 (conjugated using PTL chemistry) or 3 μg HepB core-IgE C67-8 construct. (B) Anti-receptor bound IgE reactivity of serum from Balb C mice immunised with 25 μg BSA-IgE C67-8 (conjugated using PTL chemistry) or 3 μg HepB core-IgE C67-8 construct.

FIG. 6, Competition assay with soluble IgE and IgE C67-8 peptide. Sera from BSA-IgE C67-8 or HBC-IgEC67-8 immunised mice were pre-incubated with soluble IgE (10 μg/ml) or IgE C67-8 peptide (25 μM) or the irrelevant peptide PT326 (25 μM) and added to IgE-coated ELISA plates. Data are mean±S.E.M (n=10).

FIG. 7, PT1079 Anti-IgE Data. (A) Anti-plate bound IgE reactivity of serum from Balb C mice immunised with 25 μg BSA-PT1079 (conjugated using PTL chemistry) or 3 μg HepB core-1079 construct. (B) Anti-receptor bound IgE reactivity of serum from Balb C mice immunised with 25 μg BSA-1079 (conjugated using PTL chemistry) or 3 μg HepB core-1079 construct.

FIG. 8, Competition assay with soluble IgE and PT1079 peptide. Sera from BSA-1079 or HBC-1079 immunised mice were pre-incubated with soluble IgE (10 μg/ml) or PT1079 peptide (25 μM) or the irrelevant peptide PT326 (25 μM) and added to IgE-coated ELISA plates. Data are mean±S.E.M (n=10).

FIG. 9, PT1078 Anti-IgE Data (A) Anti-plate bound IgE reactivity of serum from Balb C mice immunised with 25 μg BSA-PT1078 (conjugated using PTL chemistry. (B) Anti-receptor bound IgE reactivity of serum from Balb C mice immunised with 25 μg BSA-1078 (conjugated using PTL chemistry).

FIG. 10, Competition assay with soluble IgE and PT1078 peptide. Sera from BSA-1078 immunised mice were pre-incubated with soluble IgE (10 μg/ml) or PT1078 peptide (25 μM) or the irrelevant peptide PT326 (25 μM) and added to IgE-coated ELISA plates. Data are mean±S.E.M (n=10).

FIG. 11, PT1079gs Anti-IgE Data. (A) Anti-plate bound IgE reactivity of serum from Balb C mice immunised with 31 μg HBC-1079gs, (B) Anti-receptor bound IgE reactivity of serum from Balb C mice immunised with 3 μg HBC-1079gs.

FIG. 12, Competition assay with soluble IgE and PT1079 peptide. Sera from HBC-1079gs immunised mice were pre-incubated with soluble IgE (10 μg/ml) or PT1079 peptide (25 μM) or the irrelevant peptide PT326 (25 μM) and added to IgE-coated ELISA plates. Data are mean±S.E.M (n=10).

FIG. 13, Inhibitory Activity of Mouse BSA-C67-8 induced Antisera. Cells from a LolP1-sensitive donor were treated with mouse serum (diluted 1/50) and then triggered to release histamine with LolP1. Data are mean±S.E.M. (n=10).

FIG. 14, Inhibitory Activity of Mouse Antisera induced by BSA-1078 and BSA 1079. Cells from a LolP1-sensitive donor were treated with mouse serum (BSA and BSA-1078 anti-sera diluted 1/50; BSA-1079 aritiserum diluted 1/1250) and then triggered to release histamine with LolP1. Data are mean±S.E.M. (n=10).

FIG. 15, Inhibitory Activity of Mouse Antisera induced by HBC-C67-8, HBC-1078, HBC-1079 and HBC-1079gs. Cells from a LolP1-sensitive donor were treated with mouse serum (HBC wild type (wt) and HBC-IgEC67-8 antisera diluted 1/50; HBC-1079 and HBC-1079gs antisera diluted 1/1250) and then triggered to release histamine with LolP1. Data are mean±S.E.M. (n=10).

FIG. 16 shows the concentration dependent binding of antibody PTmAb0005 and PTmAb0011 to IgE.

FIG. 17, shows the concentration dependent inhibition of IgE binding to an FcεR1α/IgG construct with antibody PTmAb0005 and PTmAb0011 compared to control.

FIG. 18, shows the concentration dependent inhibition of IgE binding to clipped ectodomain of FcεR1α-bound directly to plastic plates, by antibody PTmAb0005, compared to control.

FIG. 19, shows IgE binding to FcεRII (CD23) by antibody PTmAb0005 (GE-1) and PTmAb0011.

FIG. 20, shows the concentration-dependent blocking of histamine release from allergic human blood basophils with antibody PTmAb0005 and PTmAb0011 compared to control.

FIG. 21, inhibition of LolP1 triggered histamine release in allergic human basophils by both PTmAb0005 and PTmAb0011.

FIG. 22, PTmAb0011 binding to different IgE; (A) PTmAb0011 Binding to Chimaeric IgE; (B) PTmAb0011 Binding to Myeloma IgE; (C) PTmAb0011 Binding to Antigen Orientated IgE; (D) PTmAb0011 Binding to Heat Denatured IgE.

FIG. 23, Inhibition of IgE Binding to FcεR1α by PTmAb0011.

FIG. 24, Binding of PTmAb0011 to Receptor Bound IgE.

FIG. 25, (A) The effect of PTmAb0011 on IgE binding to FcεRII on RPMI 8866 cells. RPMI 8866 cells (1×10⁶/ml) were incubated for an hour on ice with chimaeric IgE (1 μg/ml) and anti-IgE mAb (10 to 0 μg/ml). The IgE and anti-IgE were pre-incubated for an hour at room temperature prior to addition to the cells. Bound IgE was detected with FITC-goat anti-human IgE. The results show the mean channel fluorescence (MCF) of duplicate samples as determined by flow cytometric analysis of 10,000 live gated events. (B) Non PI specific antibody PTmAb0017.

FIG. 26, The effects of PTmAb0011 on IgE binding to FcεRII on primary human B-cells. Peripheral blood mononuclear cells (1×10⁶/ml) were incubated for an hour on ice with chimaeric IgE (1 μg/ml) and anti-IgE mAb (10 to 0 μg/ml; open) or equivalent concentrations of isotype matched control mAb (solid). The IgE and anti-IgE were pre-incubated for an hour at room temperature prior to addition to the cells. Bound IgE was detected with FITC-goat anti-human IgE and the primary B-cells were elucidated with PE-conjugated anti-CD19. The results show the mean channel fluorescence (MCF) of duplicate samples as determined by flow cytometric analysis of 5,000 live gated events.

FIG. 27, Effects of PTmAb0011 on IgE secretion from primary human B-cells. Peripheral blood mononuclear cells (2×10⁵/well) were cultured in medium supplemented with IL4 (10 ng/ml) and anti-CD40 antibody (1 μg/ml). PTmAb0011 or an isotype matched control mAb were added (1 μg/ml) for 14 days and then cell supernatant harvested and analysed for total IgE content by ELISA. The results are expressed as a percentage of the amount of IgE secreted in the absence of any antibody.

FIG. 28, Anaphylactogenicity of anti-human IgE monoclonal antibodies in allergic (A) and non-allergic (B) human basophils. PBMC from allergic donors or from non-allergic donors passively sensitised with 1 μg/ml chimeric IgE were treated with mAbs for 30 min. at 37° C. Histamine release was determined by specific EIA. Data are mean of 3 separate experiments each with different donors.

FIG. 29, Anaphylactogenicity of anti-human IgE antibodies in sensitised (A) and non-sensitised (B) human lung mast cells. Sensitised or non-sensitised crude human lung mast cell suspensions were treated with antibodies for 45 min. at 37° C. Tryptase release in supernatants was determined by calorimetric assay. Data are means of duplicate determinations from a single representative experiment.

FIG. 30, Anaphylactogenicity of anti-human IgE antibodies in RBL J41 cells through human FcεRI (A) and mouse FcεRI (B). RBL J41 cells were sensitised either with chimeric human IgE or with mouse IgE and treated with antibodies for 30 min. at 37° C. β-hexosaminidase release was determined in supernatants by calorimetric assay. Data are means of triplicate determinations from a single representative experiment. FIG. 31, Inhibition of allergen-triggered histamine release in human basophils by PTmAb0011. PBMC were incubated with PTmAb0011 either directly (allergic assay (A)) or together with IgE (blocking assay (B)) for 30 min. at 37° C. Cells were subsequently triggered with antigen for 30 min. at 37° C. and histamine release determined by specific EIA. Data are mean±s.e.m. from 3 separate experiments from different donors.

FIG. 32, Inhibition of passive cutaneous anaphylaxis in Monkey skin by PTmAb0011 and PTmAb0005. Monoclonal antibody Dec7B (stanworth decapeptide) was used as a control.

The present invention is illustrated by but not limited to the following examples.

Part 1 Mimotopes and Immunogens of the Present Invention

### EXAMPLE 1

1.1 Surface Exposed Epitope Identification, Chemical Conjugation and Serological Methods

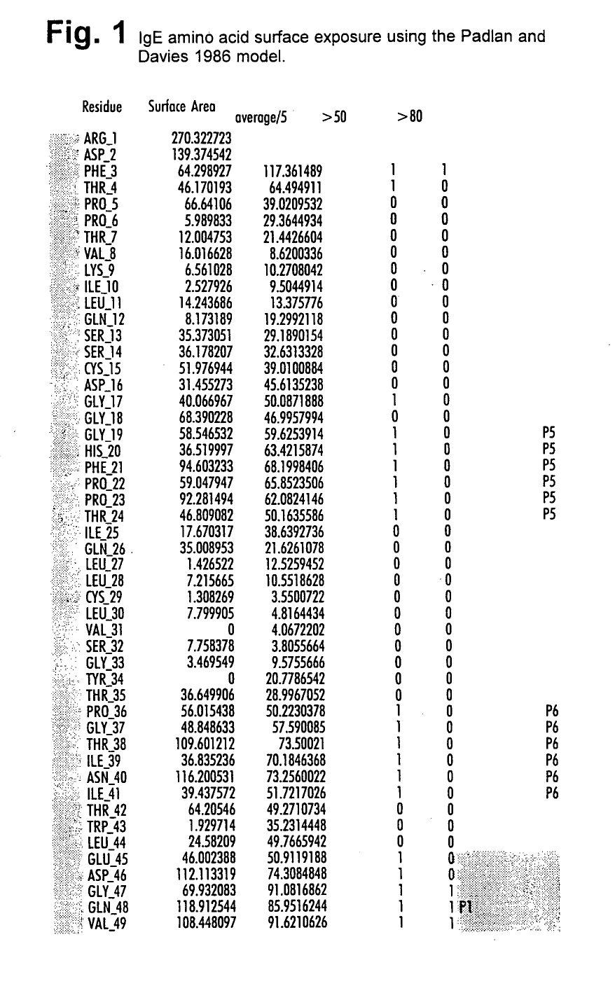

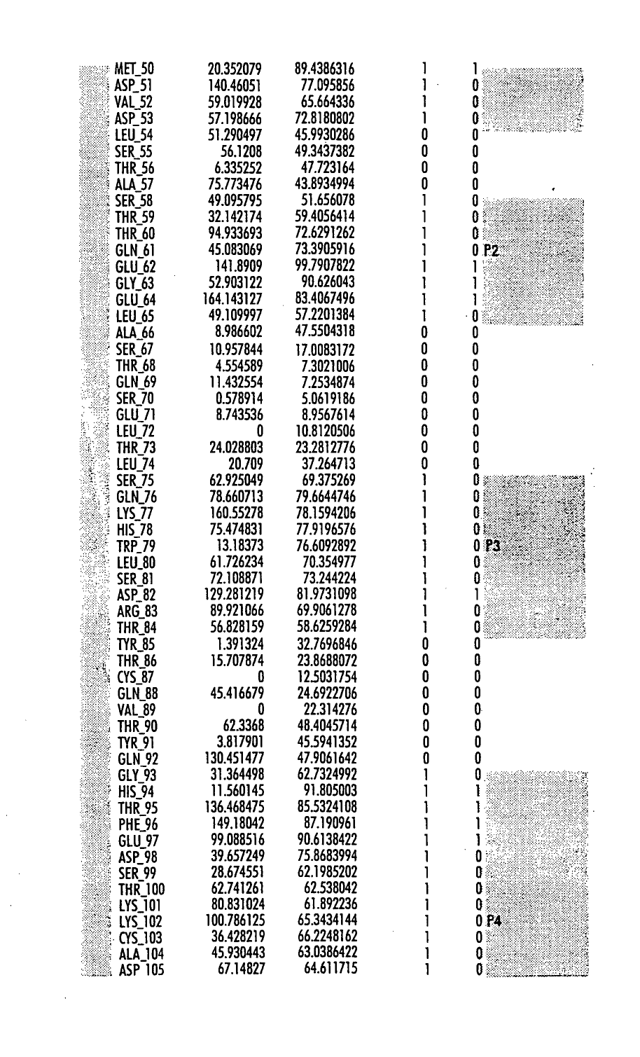

The surface exposed epitopes of the Cε2 domain of IgE were identified using the modelled structure of human IgE described by Padlan and Davies (Mol. Immunol., 23, 1063-75, 1986). Peptides were identified which were both continuous and solvent exposed. This was achieved by using Molecular Simulations software (MSI) to calculate the accessibility for each IgE amino acid, the accessible surface was averaged over a sliding window of five residues, and thereby identifying regions of the IgE peptides which had an average over that 5-mer of greater than 80 Å². The results of the test are shown in FIG. 1.

Results

From FIG. 1, and also from repeats of the same procedure using the 1990 Helm et at model (2IgE model structure deposited Feb. 10, 1990 with PDB (Protein Data Bank, Research Collabarotory for Structural Bioinformatics)), there are a number of native peptides which may be used as immunogens for raising antibodies against IgE.

TABLE 1

Native surface exposed and
continuous IgE peptides

| Peptide | | Location sequence and | SEQ |
|---|---|---|---|
| Name | Sequence | IgE Domain | ID NO. |
| P1 | EDGQVMDVD | Cε2 (Glu270-Asp278) | 1 |
| P2 | STTQEGEL | Cε2 (Ser283-Leu290) | 2 |
| P3 | SQKHWLSDRT | Cε2 (Ser300-Thr309) | 3 |
| P4 | GHTFEDSTKK | Cε2 (Gly318-Lys327) | 4 |
| P5 | GGGHFPPT | Cε2 (Gly245-Thr250) | 5 |
| P6 | PGTINI | Cε2 (Pro262-Ile267) | 6 |
| P7 | FTPPT | Cε2 (Phe231-Thr235) | 7 |

These peptides, or mimotopes thereof, were synthesised and either conjugated to carrier proteins, or put into Hepatitis Core antigen constructs to form recombinant peptide expressing virus-like particles.

1.2 Synthesis of IgE Peptide/Protein D Conjugates Using a Succinimide-Maleimide Cross-Linker

Protein D may be conjugated directly to IgE peptides to form antigens of the present invention by using a maleimide-succinimide cross-linker. This chemistry allows controlled NH₂ activation of carrier residues by fixing a succinimide group. The Maleimide group is a cysteine-binding site. Therefore, for the purpose of the following examples, the IgE peptides to be conjugated require the addition of an N-terminal cysteine.

The coupling reagent is a selective heterobifunctional cross-linker, one end of the compound activating amino group of the protein carrier by an succinimidyl ester and the other end coupling sulhydryl group of the peptide by a maleimido group. The reaction scheme is as the following:
a. Activation of the protein by reaction between lysine and succinimidyl ester: 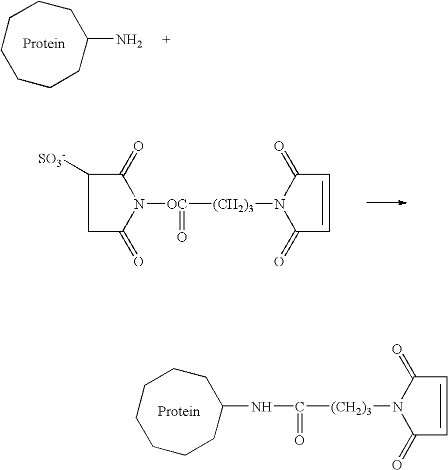
b. Coupling between activated protein and the peptide cysteine by reaction with the maleimido group: 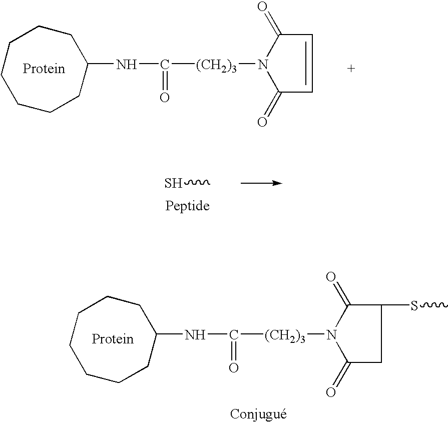
   1.3 Preparation of IgE Peptide-Protein D Conjugate

The protein D is dissolved in a phosphate buffer saline at a pH 7.2 at a concentration of 2.5 mg/ml. The coupling reagent (N-[γ-maleimidobutyryloxy] succinimide ester—GMBS) is dissolved at 102.5 mg/ml in DMSO and added to the protein solution. 1.025 mg of GMBS is used for 1 mg of Protein D. The reaction solution is incubated 1 hour at room temperature. The by-products are removed by a desalting step onto a sephacryl 200HR permeation gel. The eluant used is a phosphate buffer saline Tween 80 0.1% pH 6.8. The activated protein is collected and pooled. The peptides (as identified in table 1 or derivatives or mimotopes thereof) is dissolved at 4 mg/ml in 0.1 M acetic acid to avoid di-sulfide bond formation. A molar ratio of between 2 to 20 peptides per 1 molecule of activated Protein D is used for the coupling. The peptide solution is slowly added to the protein and the mixture is incubated 1 h at 25° C. The pH is kept at a value of 6.6 during the coupling phase. A quenching step is performed by addition of cysteine (0.1 mg cysteine per mg of activated PD dissolved at 4 mg/ml in acetic acid 0.1 M), 30 minutes at 25° C. and a pH of 6.5. Two dialyses against NaCl 150 mM Tween 80 0.1% are performed to remove the excess of cysteine or peptide.

The last step is sterilising filtration on a 0.22 μm membrane. The final product is a clear filtrable solution conserved at 4° C. The final ratio of peptide/PD may be determined by amino acid analysis.

In an analogous fashion the peptides of the present invention may be conjugated to other carriers including BSA.

A mimotope of P1 was synthesised CLEDGQVMDVDLL (P15, SEQ ID NO. 8) which was conjugated to both Protein D and BSA using techniques described above.

1.4 ELISA Methods

Anti-Peptide or Anti-Peptide Carrier ELISA

The anti-peptide and anti-carrier immune responses were investigated using an ELISA technique outlined below. Microtiterplates (Nunc) are coated with the specific antigen in PBS (4° overnight) with either: Streptavidin at 2 μg/ml (followed by incubation with biotinylated peptide (1 μM) for 1 hour at 37° C.), Wash 3×PBS-Tween 20 0.1%. Saturate plates with PBS-BSA 1%-Tween 20 0.1% (Sat buffer) for 1 hr at 37°. Add 1° antibody=sera in two-step dilution (in Sat buffer), incubate 1 hr 30 minutes at 37°. Wash 3×. Add 2° anti-mouse Ig (or anti-mouse isotype specific monoclonal antibody) coupled to HRP. Incubate 1 hr at 370. Wash 5×. Reveal with TMB (BioRad) for 10 minutes at room temperature in the dark. Block reaction with 0.4N H₂SO₄.

Method for the Detection of Anti-Human IgE Reactivity in Mouse Serum (IgE Plate Bound ELISA)

ELISA plates are coated with human chimaeric IgE at 1 μg/ml in pH 9.6 carbonate/bicarbonate coating buffer for 1 hour at 37° C. or overnight at 4° C. Non-specific binding sites are blocked with PBS/0.05% Tween-20 containing 5% w/v Marvel milk powder for 1 hour at 37° C. Serial dilutions of mouse serum in PBS/0.05% Tween-20/1% w/v BSA/4% New Born Calf serum are then added for 1 hour at 37° C. Polyclonal serum binding is detected with goat anti-mouse IgG-Biotin (1/2000) followed by Streptavidin-HRP (1/1000). Conjugated antibody is detected with TMB substrate at 450nm. A standard curve of PTmAb0011 is included on each plate so that the anti-IgE reactivity in serum samples can be calculated in μg/ml.

Method for the Detection of Anti-Human Receptor-Bound IgE Reactivity in Mouse Serum

ELISA plates are coated with recombinant human FεFR1α at 0.5 μg/ml in pH 9.6 carbonate/bicarbonate coating buffer for 1 hour at 37° C. or overnight at 4° C. Non-specific binding sites are blocked with PBS/0.05% Tween-20 containing 5% w/v Marvel low fat milk powder for 1 hour at 37° C. Human IgE at 1 μg/ml is then added for 1 hour at 37° C. Serial dilutions of mouse serum in PBS/0.05% Tween-20/1% w/v BSA/4% New Born Calf serum are then added for 1 hour at 37° C. Polyclonal serum binding is detected with goat anti-mouse IgG-Biotin (1/2000) followed by Streptavidin-HRP (1/1000). Conjugated antibody is detected with TMB substrate at 450 nm. A standard curve of PTmAb0011 is included on each plate so that the anti-IgE reactivity in serum samples can be calculated in μg/ml.

Competition of IgE Binding with Mimotope Peptides, Soluble IgE or PTmAb0011

Single dilutions of polyclonal mouse serum are mixed with single concentrations of either mimotope peptide or human IgE in a pre-blocked polypropylene 96-well plate. Mixtures are incubated for 1 hour at 37° C. and then added to IgE-coated ELISA plates for 1 hour at 37° C. Polyclonal serum binding is detected with goat anti-mouse IgG-Biotin (1/2000) followed by Streptavidin-HRP (1/1000). Conjugated antibody is detected with TMB substrate at 450 nm. For competition between serum and PTmAb0011 for IgE binding, mixtures of serum and PTmAb0011-biotin are added to IgE-coated ELISA plates. PTmAb0011 binding is detected with Streptavidin-HRP (1/1000).

1. 5 Human Basophil Assays

Two types of assay were performed with human basophils (HBA), one to determine the anaphylactogenicity of the monoclonal antibodies, consisting of adding the antibodies to isolated PBMC; and a second to measure the inhibition of LolP1 (a strong allergen) triggered histamine release be pre-incubation of the HBA with the monoclonal antibodies.

Blood is collected by venepuncture from allergic donors into tubes containing 0.1 volumes 2.7% EDTA, pH 7.0. It is then diluted 1/2 with an equal volume of HBH medium containing 0.1% human serum albumin (HBH/HSA). The resulting cell suspension is layered over 50% volume Ficoll-Paque and centrifuged at 400 g for 30 minutes at room temperature. The peripheral blood mononuclear cell (PBMC) layer at the interface is collected and the pellet is discarded. The cells are washed once in HBH/HSA, counted, and re-suspended in HBH/HSA at a cell density of 2.0×10⁶ per ml. 100 μl cell suspension are added to wells of a V-bottom 96-well plate containing 100 μl diluted test sample or monoclonal antibody. Each test sample is tested at a range of dilutions with 6 wells for each dilution. Well contents are mixed briefly using a plate shaker, before incubation at 37° C. for 30 minutes with shaking at 120 rpm.

For each serum dilution 3 wells are triggered by addition of 10 μt LolP1 extract (final dilution 1/10000) and 3 wells have 10 μl HBH/HSA added for assessment of anaphylactogenicity. Well contents are again mixed briefly using a plate shaker, before incubation at 37° C. for a further 30 minutes with shaking at 120 rpm. Incubations are terminated by centrifugation at 500 g for 5 min. Supernatants are removed for histamine assay using a commercially available histamine EIA measuring kit (Immunotech). Control wells containing cells without test sample are routinely included to determine spontaneous and triggered release. Wells containing cells+0.05% Igepal detergent are also included to determine total cell histamine.

The results are expressed as following:

Anaphylactogenesis Assay
Histamine release due to test samples=% histamine release from test sample treated cells−% spontaneous histamine release.
Blocking Assay

The degree of inhibition of histamine release can be calculated using the formula:

$$\% inhibition = 1 - \frac{( \begin{matrix}histamine release from test \\ sample treated {cells}^{*}\end{matrix} )}{( \begin{matrix}histamine release from antigen \\ stimulated {cells}^{*}\end{matrix} )} \times 100$$

Values corrected for spontaneous release.

### EXAMPLE 2

### Immunisation of Mice with P15 Conjugates (P15-BSA or P15-PD) Induces Production of Anti-Human IgE Antibodies

The conjugates comprising the mimotope P15 (25 μg protein/dose), described in 1.4, were administered into groups of 10 BalbC mice, adjuvanted with and oil in water emulsion containing QS21 and 3D-MPL described in WO 95/17210 . Boosting was be performed on day 21 and on day 42 and sera can be harvested on day 42 and 56. The immune response anti-peptide and anti-plate bound IgE was followed using methods described in Example 1.

Results

The results for anti peptide and anti-[gE responses measured at day 14 post third vaccination are shown in table 2.

TABLE 2
P15 Immnuogenicity results

| | Anti-peptide responses | | | Anti-IgE responses | | |
|---|---|---|---|---|---|---|
| | (Mid point titre) | | | (μg/ml (PTmAb0011)) | | |
| Mimotope | | Std | | | Std | |
| conjugate | Average | Dev. | Geomean | Average | Dev. | Geomean |
| P15-PD | 41391 | 26858 | 36154 | 1.6 | 4.5 | 0.3 |
| (n = 16) | | | | | | |
| P15-BSA | 49591 | 9259 | 48719 | 2.2 | 2.5 | 1.0 |
| (n = 10) | | | | | | |

### EXAMPLE 3

### Anti-IgE Induced in Mice After Immunisation with Conjugate are Non Anaphylactogenic

Several dilutions of complete sera or IgG purified from conjugate immunised mice can be tested in presence of basophils from freshly harvested peripheral blood from allergic patients.

The anaphylactogenicity can be evaluated by the measuring of the histamine released induced by the antibodies to be tested as described below:
Erythrocytes are removed from peripheral blood on glucose dextran gradient
Cells are washed and plated with samples to be tested (for example allergen, antibodies, allergen plus antibodies, . . . )
After incubation, supernatants are collected and histamine release is measured according to manufacturer's instructions (Immunotech, histamine enzyme immunoassay kit)
   Neither antiserum generated with P15-BSA or P15-PD was shown to be anaphylactogenic.

### EXAMPLE 4

### Anti-IgE Induced in Mice After Immunisation with Conjugate are Capable of Blocking IgE Mediated Histamine Release Induced by Allergen Triggering of Basophil from Allergic Patient

Histamine release can be measured in basophil samples triggered with various concentrations of allergen in presence or absence of several dilutions of complete sera or IgG purified from conjugate immunised mice. Blocking activity of anti-P15 antibodies in the antiserum was evaluated by the measuring of the inhibition of the histamine release induced by the allergen. Histamine release and inhibition was measured as described in example 3. As P15 is a mimotope of P1, PTmAb0011 was used as a control as it is known to bind to the same epitope (P1). The results are shown in table 3.

TABLE 3
Histamine release inhibition from allergic human basophils

| | | % inhibition of |
|---|---|---|
| Antiserum | Dilution | histamine release |
| P15-PD (mouse 4.12) | 1/30 | 79 |
| P15-PD (mouse 4.5) | 1/30 | 57 |
| P15-BSA (mouse 7.3) | 1/30 | 67 |
| P15-BSA (mouse 7.5) | 1/30 | 57 |
| PTmAb0011 | 0.1 μg/ml | 56 |
| PTmAb0011 | 1 μg/ml | 90 |
| anti-BSA serum | 1/30 | 40 |
| anti-PD serum | 1/30 | 40 |

### EXAMPLE 5

### Immunogenicity of Mimotopes of P2 and P3

The following mimotopes were conjugated to BSA using techniques described in example 1.2, and mice were immunised with the conjugates using the same formulation and schedule as that described in example 2.

| | | | |
|---|---|---|---|
| P16 | CSTTQEGELA-NH₂ | P2 mimotope | SEQ ID NO.24 |
| P17 | CSQKHWLSDRT-NH₂ | P3 mimotope | SEQ ID NO.26 |

The mice were bled after the last immunisation and tested for anti-IgE reactivity in the IgE plate bound ELISA. The individual, average (Av), geomean (GM), results are summarised below (SD=standard deviation).

TABLE 4
P16 and P17 immunogenicity results

| | Anti peptide immune responses/mouse | | | | | | | | | | | | |
|---|---|---|---|---|---|---|---|---|---|---|---|---|---|
| | (14 days after third vaccination), Mid point titres. | | | | | | | | | | | | |
| | 1 | 2 | 3 | 4 | 5 | 6 | 7 | 8 | 9 | 10 | Av | SD | GM |
| P16 | 1891 | 649 | 1299 | 2349 | 591 | 1474 | 4605 | 918 | 4177 | 865 | 1882 | 1436 | 1478 |
| P17 | 100 | 4349 | 2850 | 3434 | 6133 | 2231 | 5085 | 2991 | 13070 | 8874 | 5446 | 3515 | 4656 |

### EXAMPLE 6

### Production of Mimotopes of P1, and Immunogenicity/Functional Activity Thereof

6.1 Production of Immunogens

Mimotopes of P1 were derived either by phage display techniques or by rational design by molecular modelling of the C-D loop of Cε2 domain of IgE. The following peptides were synthesised and formulated into both BSA-peptide conjugates and also into HepB core antigen recombinant constructs.

| | | |
|---|---|---|
| Name of | | |
| peptide | Sequence | SEQ ID NO. |

| | | | |
|---|---|---|---|
| C67/8 | CFINKQMADLELCPRE | P1 mimotope | 12 |
| PT1079 | CLEDGQVMDVDLCPREAAEGD | P1 mimotope | 14 |
| PT1079GS | CLEDGQVMDVDLCGGSSGGP | P1 mimotope | 15 |
| PT1078 | CLEDGQVMDVDCPREAAEGDK | P1 mimotope | 16 |

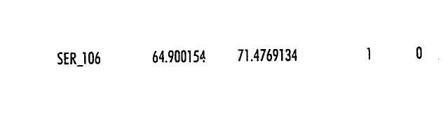

The peptides/protein carrier constructs were produced as follows. Acylhydrazine peptide derivatives were prepared on the solid phase as shown in scheme 1 (FIG. 2). These peptide derivatives can be readily prepared using the well-known ‘Fmoc’ procedure, utilising either polyamide or polyethyleneglycol-polysiyrene (PEG-PS) supports in a fully automated apparatus, through techniques well known in the art [techniques and procedures for solid phase synthesis are described in ‘Solid Phase Peptide Synthesis: A Practical Approach’ by E. Atherton and R. C. Sheppard, published by IRL at Oxford University Press (1989)]. Acid mediated cleavage afforded the linear, deprotected, modified peptide. This could be readily oxidised and purified to yield the disulphide-bridged modified epitope using methodology outlined in ‘Methods in Molecular Biology, Vol. 35: Peptide Synthesis Protocols (ed. M. W. Pennington and B. M. Dunn), chapter 7, pp9l-171 by D. Andreau et al.

The peptides thus synthesised can then be conjugated to protein carriers (in this case Bovine Serum Albumin, BSA) using the following technique:

6.2 Modified Carrier Synthesis

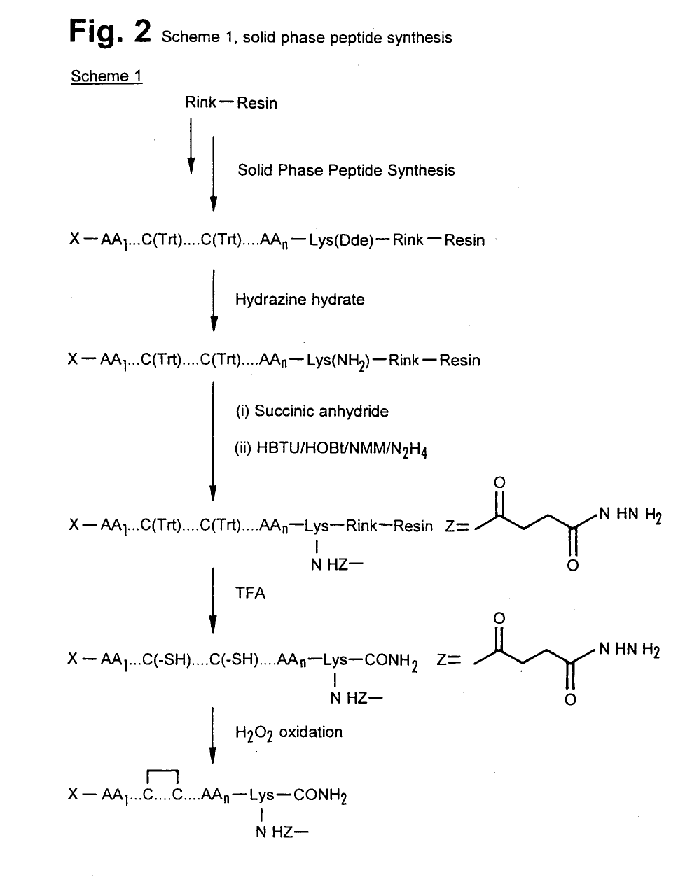

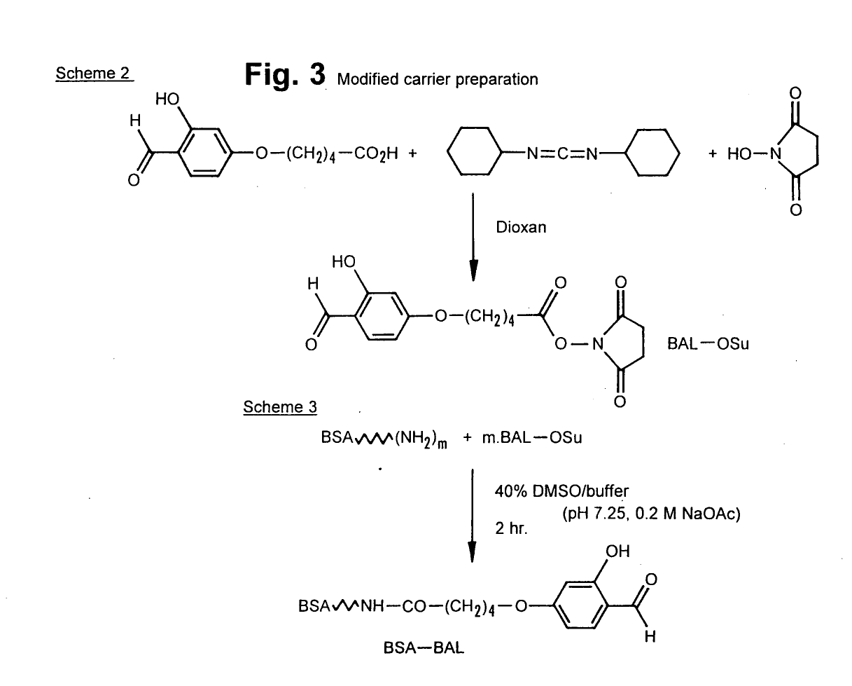

Introduction of the aryl aldehyde functionality utilised the succinimido active ester (BAL-OSu) prepared as shown in scheme 2 (FIG. 3, see WO 98/17628 for further details). Substitution of the amino functions of BSA (bovine serum albumin) to ˜50% gave routinely soluble modified protein. Greater substitution of the BSA led to insoluble constructs. BSA and BAL-OSu were mixed in equimolar concentration in DMSO/buffer (see scheme 3, FIG. 3) for 2 hrs. This experimentally derived protocol gave ˜50% substitution of BSA as judged by the Fluorescamine test for free amino groups.

6.3 Peptide-BSA Construct:

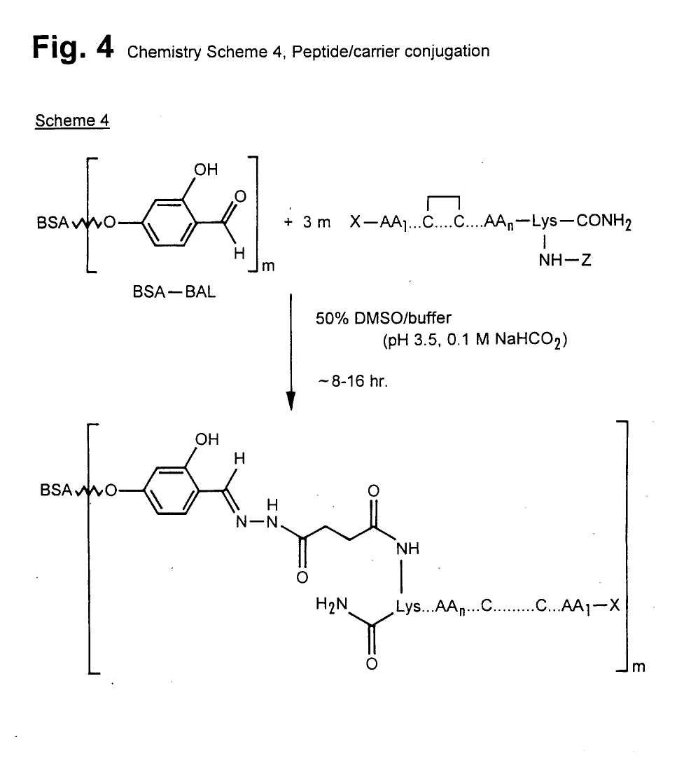

Simple combination of modified peptide and derivatised BSA afforded peptide-BSA constructs readily isolated by dialysis (scheme 4, FIG. 4). SDS-PAGE was used to confirm an increase in molecular weight.

6.4 Hepatitis Core Antigen Constructs

Hepatitis Core antigen recombinant constructs (HBC) were also prepared, using lo molecular biology techniques described in EP 0 421 635 B. In these HBC experiments PT1079 was modified to remove the terminal lysine.

| | | |
|---|---|---|
| Peptide | Sequence | SEQ ID NO. |

| | | |
|---|---|---|
| PT1078HBC | CLEDGQVMDVDCPREAAEGD | 65 |
| PT1079HBC | CLEDGQVMDVDLCPREAAEGD | 66 |

The expression of the P1 -mimotope peptides was confirmed by BIAcore experiments with PTmAb0005 and PTmAb0011. The immunogenicity results were generated using doses of only 3 μg/dose of HBC.

6.5 Immunogenicity Studies

The mimotope/HBC and mitnotope/BSA constructs were purified and formulated into vaccines and adjuvanted with an oil in water emulsion containing QS21 and 3D-MPL described in WO 95/17210 the (25 μg BSA conjugate dose). These vaccines were administered into groups of 10 BalbC mice, and boosting was be performed on day 14 and on day 28 and sera was harvested on day 42. The immune response to anti-plate bound IgE and receptor orientated IgE, was then followed using the techniques described in example 1.4. Also, the activity of the antiserum in the inhibition of histamine release from allergic basophils was measured in the techniques described in 1.5.

6.6 Results

All BSA and HBC constructs induced high titres of anti-IgE antibodies, when the IgE was bound directly to the ELISA plate, and when orientated on the high affinity receptor. Moreover, all of these responses were confirmed to be specific, in that they were competed by free IgE and the mimotope itself, and not by non-specific peptides. The anti-IgE induced by these immunogens were capable of inhibiting histamine release from human basophils derived from an allergic donor (rye grass, LOLP1).

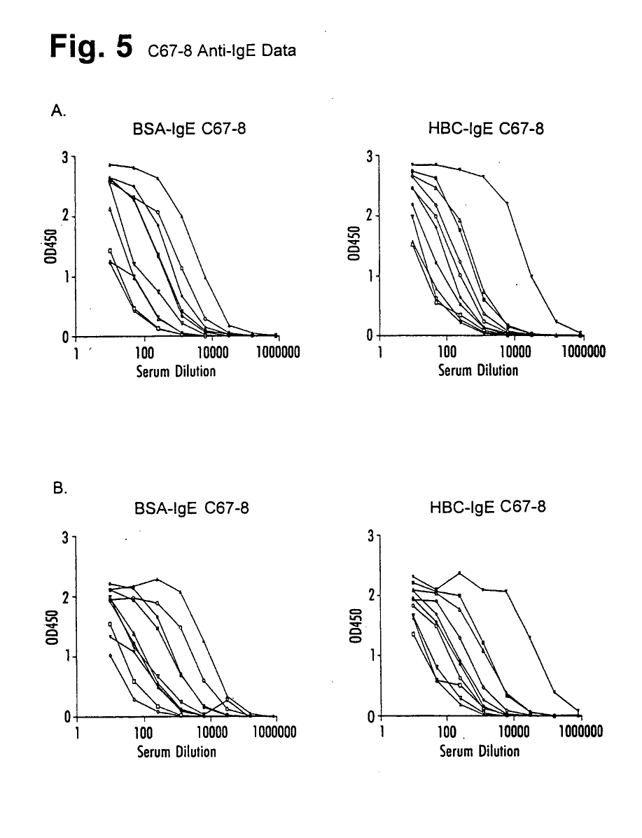

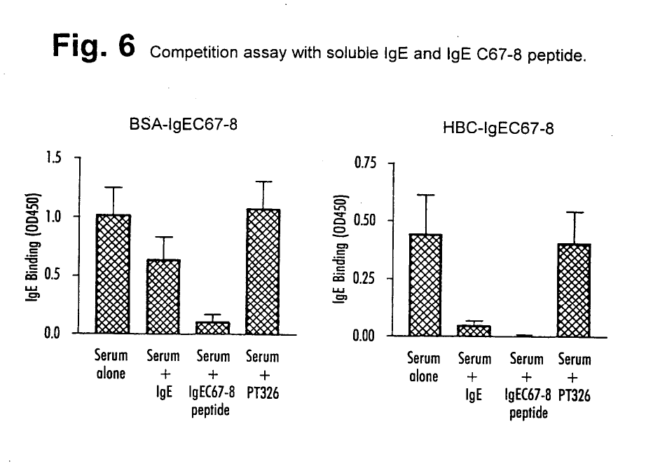

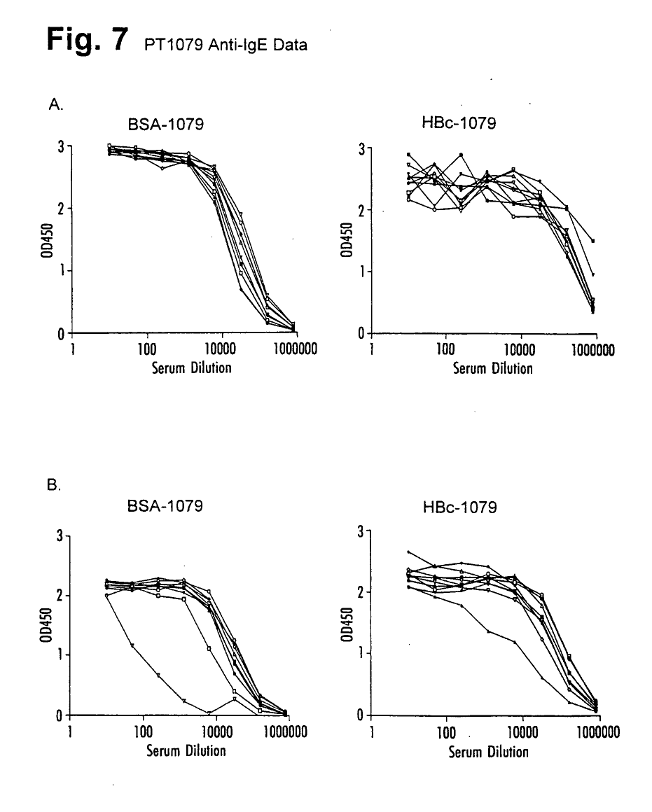

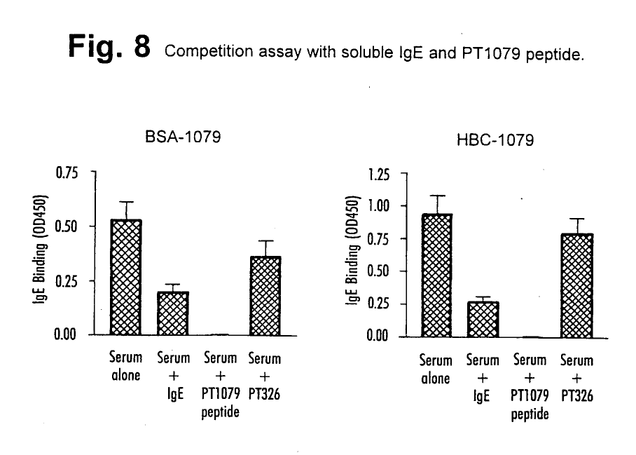

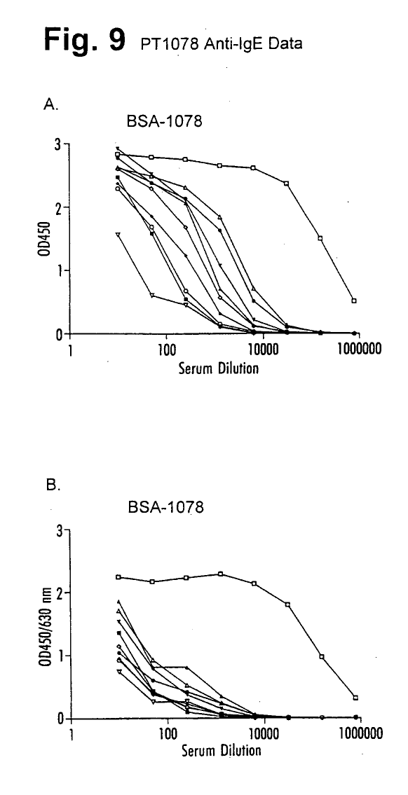

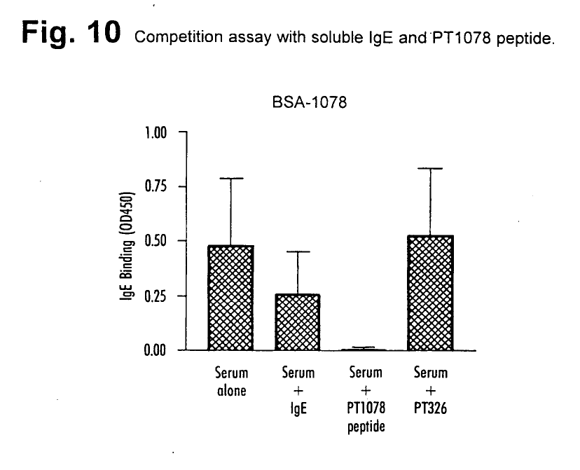

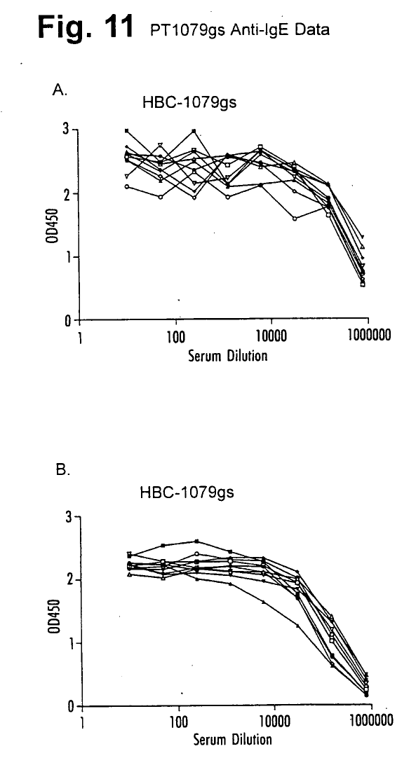

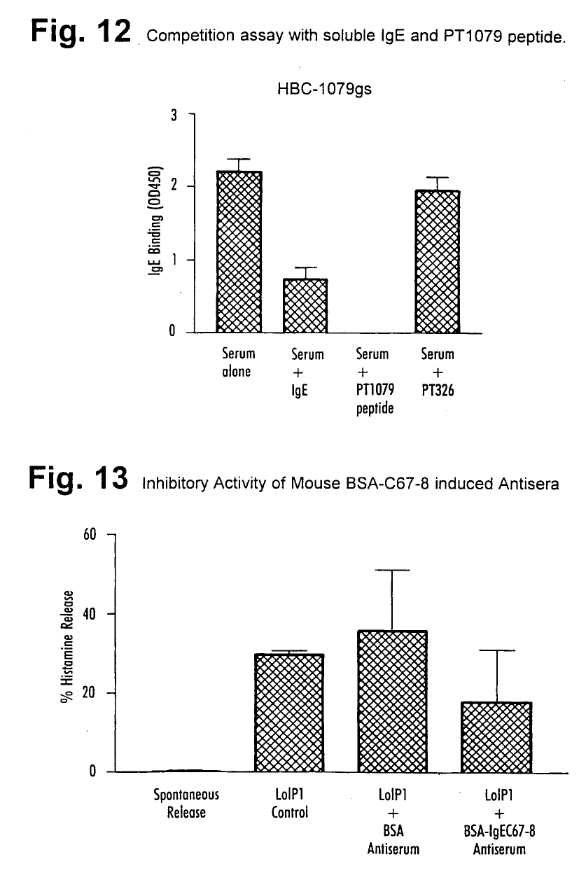

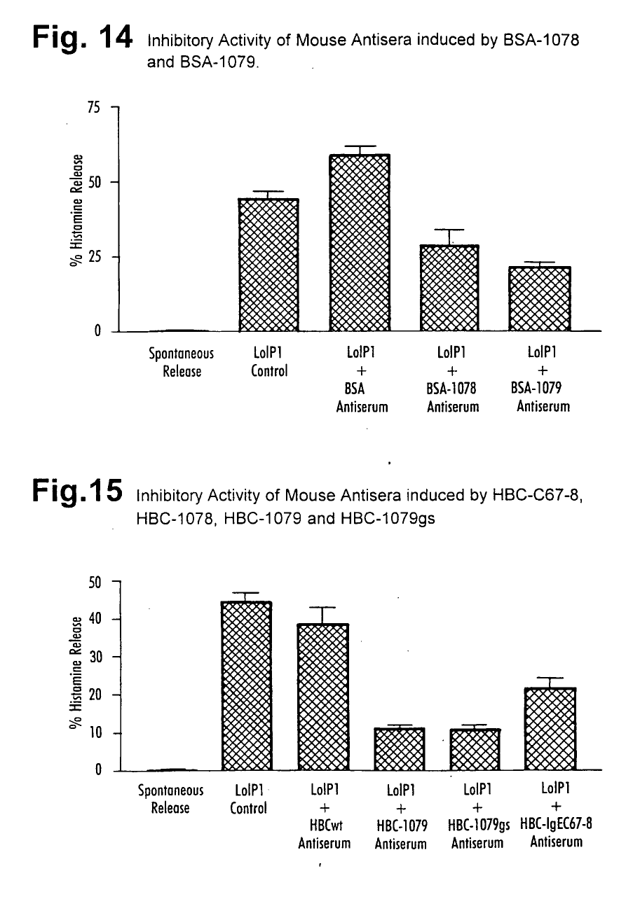

For the results for C67-8 see FIGS. 5, 6, 13 and 15. For the results for PT1078 see FIGS. 9, 10, 14 and 15. For the results for PT1079 see FIGS. 7, 8, 14 and 15. For the results for PT1079GS see FIGS. 11, 12 and 15.

Moreover, the immune responses generated by these peptide mimotopes were not anaphylactogenic.

TABLE 5
Anaphylactogenicity of the P1 mimotope antisera

| | Serum | % Histamine |
|---|---|---|
| Immunogen | Dilution | Release |
| Spontaneous Release | | 0.25 ± 0.06 |
| Naïve serum | 1/50 | 1.9 ± 0.4 |
| BSA | 1/50 | 2.15 ± 0.65 |
| BSA-IgE C67-8 | 1/50 | 2.9 ± 1.1 |
| BSA-1078 | 1/50 | 5.00 ± 1.40 |
| BSA-1079 | 1/1250 | 0.43 ± 0.04 |
| HBCwt | 1/50 | 3.5 ± 1.0 |
| HBC-1079 | 1/1250 | 0.12 ± 0.04 |
| HBC-1079gs | 1/1250 | 0.02 ± 0.02 |
| HBC-IgE C67-8 | 1/50 | 2.14 ± 0.26 |

Footnote to table,
Cells from a LolP1-sensitive donor were treated with diluted mouse serum for 30 mins. Released histamine was determined by a commercially available histamine specific EIA.
Data are mean ± S.E.M. (n = 10).

Part 2 Ligands that Bind to the Epitopes and Mimotopes of the Present Invention

Peptide immunogens are described in part 1, which after administered to a mammal in the form of a vaccine, induce immune responses which (a) recognise IgE, and (b) are capable of inhibiting histamine release in vitro. Part 2 describes ligands that are capable of binding to the epitopes or mimotopes of the present invention, and describes their function. Two monoclonal antibodies have been identified, PTmAb0005 and PTmAb0011, which recognise the c-d loop of Cε2 of IgE. Mimotopes of this peptide have been shown in part 1 to be immunogenic and functional in active vaccination. This section describes the characterisation of these monoclonal antibodies and provides evidence of their utility in passive vaccination.

The target epitope of the antibodies was identified using phage panning techniques, namely sequence alignment of multiple bacteriophage targets, and subsequently refined and confirmed by domain mapping and site directed mutagenesis. The functional activity of the antibodies has been confirmed not only in vitro by assaying for anti-IgE recognition and inhibition of allergic mediator release; but also in vivo in monkey Passive Cutaneous Anaphylaxis (PCA) studies.

### EXAMPLE 7

7.1 Phage Mapping of Monoclonal Antibody Target

Phage display libraries were used to map the binding sites of the monoclonal antibodies using three different phage libraries, displaying either the XCX₁₅, XCX₁₀ or XAX₁₀ peptide sequence (where X is any amino acid) at the N-terminus of the phage gVIIIp. Tables 6 and 7 show the results of selecting for peptide ligands with the anti human IgE monoclonal antibodies PTmAb0005 and respectively. Amino acid pattern similarities between the peptides and human IgE revealed a strong homology match with the c-d loop in the Cε2 domain of IgE. The homology pattern produced from the phage returns was: Q h h a h a h (where h=hydrophobic amino acid and a=acidic amino acid) and this aligned to the sequence QVMDVDL (SEQ ID NO. 17) in the C-D loop of the human IgE Cε2 domain.

IgEC67, the peptide derived from phage panning experiments and which had the highest affinity to PTmAb0005 was also epitope mapped. This was performed by introducing random mutations by PCR mutagenesis and sub-cloning into the Fuse 5 vector for minor filamentous phage protein gIIIp display. The IgEC67 mutants were ranked in order of binding to PTmAb0005, as shown in Table 8. These, and other results, confirmed the importance of the amino acids within IgEC67 which aligned with the Cε2 epitope. For example the L8P, D10G, L11M, E12G and L13R mutants all reduced binding to the anti-IgE PTmAb0005 (Data not shown). Mutations in other sites had little effect on the affinity to PTmAb0005.

Random sub-libraries were made of the highest affinity PTmAb0005 and PTmAb0011 phage display derived peptides to enhance the affinity of the peptide to the antibodies by adapting methods described previously (Yu, J. and Smith, G. P. (1996) “Affinity maturation of phage-displayed peptide ligands.” Methods in Enzymology, 267, 3-27). This involved a DNA sub-cloning transfer from the major coat protein (gVIIIp) filamentous phage display vector to a lower copy number minor phage coat protein (gIIIp) display vector with a random PCR step. Sub-libraries were made of several phage sequences including the highest affinity PTmAb0005 ligand IgEC67 and IgE C67-8. Affinity matured sequences for C67 and C67-8 are shown in tables 8 and 9 respectively. Included in the tables are ranking orders and also BIAcore affinities where available. IgEC67-8 capable of inducing an anti-human IgE response in mice when the peptide expressing phage was used as an immunogen.

7.2 Confirmation of Target by Domain Mapping

A number of constructs were generated in order to map the binding specificities of PTmAb0005 and PTmAb0011 with respect to the IgE constant domains. The following constructs were generated: Cε2-4, Cε2-3, Cε3-4, Cε3-4L (Cε3-4 plus linker sequence between domains Cε2 and Cε3) and Cε2 alone.

Fragments encompassing various domain(s) of human IgE Fc were cloned using cDNA derived from the hybridoma line JW8/5/13, which expresses a chimaeric human IgE (Neuberger, MS et al (1985) Nature 314 268-270; Bruggemann, M et al (1987) J Exp Med 166 1351-61). The IgE Fc fragments were amplified using appropriate primer pairs and JW8/5/3 cDNA as template. The cε2-4 fragment encodes amino acids (aa) S225-K547. The cε3-4 fragment encodes aa G335-547. The cε3-4L fragment (domains 3-4 plus the linker sequence that joins cε2 to cε3) encodes aa E322-K547. The cε2-3 fragment encodes aa S225-G436. The cε2 fragment encodes aa S225-S324. All constructs contain a COOH terminal hexahistidine tail for detection and purification purposes. These fragments were cloned into a eukaryotic expression vector in frame with a CD33 derived leader encoding sequence to direct secretion of the expressed fragment. This enabled expression in mammalian cell lines. The vector was derived from pcDNA3.1+ (Invitrogen). To express the cloned fragments, the appropriate clones were transfected into COS-7 cells and the resulting conditioned medium harvested 48-60 hours post transfection.

The binding of PTmAb0005 and PTmAb0011 to the expressed IgE domains was investigated by ELISA assay, by binding the constructs to an ELISA plate followed by incubation with the monoclonal antibodies, and revelation with an anti-mouse antibody. Also, binding to denatured constructs was investigated by the well known technique of Western blot.

The results for PTmAb0005 showed strong binding to Cε2-4, Cε2-3 and Cε2 in their native forms, and also bound to Cε24 and Cε2 after denaturation in western blot. No binding to Cε3-4 or Cε3-4L was observed in either assay. PTmAb0011 also bound to Cε24, Cε2-3 and Cε2 in their native form; and also bound to Cε2-4 and Cε2 in their denatured forms.

It is clear therefore that both antibodies recognised a target epitope present in the Cε2 domain of IgE.

7.3 Confirmation of Target by Site Directed Mutagenesis

Domain mapping studies demonstrated that both mAbs were able to bind to the Cε2 domain alone. Analysis of sequences derived from biopanning of phage displayed peptide libraries revealed that PTmAb0005 derived sequences showed striking similarity to P1. This region forms a loop between the C-D β strands of Cε2 in the IgE model structure. Site-directed mutagenesis studies were undertaken to validate this sequence as the epitope for PTmAb0005 and PTmAb0011.

Analysis of the panned phage sequences and a comparison of the IgE model structure (Helm et al 1990, supra) with the known structure of human IgG1 Fc (Deisenhoffer, J., 1981, Biochemistry, 20, 2361-2370) led to the identification of three residues that were likely to be involved in antibody recognition. These residues are glutamine (Q) 273, methionine (M) 275 and aspartate (D) 276. Each of these was changed to alanine (A) and at least one other amino acid residue as shown below.
Q273: A and E (glutamate)
M275: A; Q and K (lysine)
D276: A and N (asparagine)

The alanine mutations changed both the structure and chemistry of the target residue whilst the other mutations maintained structure (as closely as possible) but altered the charge, e.g Q273E. Here, glutamate has essentially the same structure as glutamine but is negatively charged instead of neutral.

Each mutation was generated independently in a Cε2-4 construct. Each mutant polypeptide was expressed to a similar level as the wild type (WT) Cε2-4 and each was able to bind to recombinant FcεRIα ectodomain as efficiently as the WT cε2-4 product in ELISA based assays. Together, these data demonstrated that the mutations had no effect on the production/secretion of the polypeptides in the expression system and did not grossly affect the structure of the cε2-4 fragment.

All mutations essentially abrogated binding to both PTmAb0005 and PTmAb0011 except D276N which reduced binding to PTmAb0005 by only 50% (Table 10). Mutation of an alternative glutamine residue within Cε2, Q317, was carried out to act as a control in these experiments. Q317E and Q317K mutants were generated and found to have no affect on the ability of PTmAb0005 and PTmAb0011 to recognise Cε2-4. Similarly, recognition of FcεR1α was not affected.

Thus, the binding activities of PTmAb0005 and PTmAb0011 are specifically affected by mutations within the C-D loop of Cε2.

In summary, the sequence PI comprises the major binding determinant for both PTmAb0005 and PTmAb0011.

TABLE 10
recognition of IgE domain constructs by PTmAb0005 and PTmAb0011.

| | | | Recognition by |
|---|---|---|---|
| | Recognition by | Recognition by | FcεR1α |
| Mutation | PTmAb0005 | PTmAb0011 | ectodomain |
| WT cε2-4 | ++++ | ++++ | ++++ |
| Q273A | − | − | ++++ |
| Q273E | − | − | ++++ |
| M275A | +/− | +/− | ++++ |
| M275Q | +/− | +/− | ++++ |
| M275K | +/− | +/− | ++++ |
| D276A | − | − | ++++ |
| D276N | ++ | − | ++++ |
| Q317E | ++++ | ++++ | ++++ |
| Q317K | ++++ | ++++ | ++++ |

7.4 Refined Modelling of the C-D Loop of IgE Cε2

As the exact structure of human IgE has not been determined yet (although a model is available) there are likely to be errors in this model structure after inspection at a detailed level. The present inventors, therefore, have refined this model of the Cε2 loop region of IgE by mapping this loop onto the equivalent region of Cγ2 of human IgG1 (Deisenhoffer J 1981 supra).

From this new information about the confines of the structural features, a cyclised peptide was designed which when synthesised should adopt a conformation which closely resembles that of the C-D loop of Cε2 in the context of the full IgE molecule.

This peptide, Ac-CLEDGVQMDVDLCPREAAEGDK(Ac)-NH₂, was named PT1079 (SEQ ID NO. 14).

The affinity of PT1079 to both PTmAb0005 and PTmAb0011 was measured using a BIAcore technique and was found to exhibit very strong recognition to both of these monoclonal antibodies (recognised by both PTmAb0005 and PTmAb0011 with apparent affinities of 20 nM and-250 nM respectively). Control, derivative peptides of PT1079, where the site of cyclisation was shifted by only one amino acid residue, thereby decreasing the length of peptide between the cyclisation sites by one amino acid residue (PT1078), reduced the binding of the peptide to either PTmab0005 or PTmAb0011. Also, PT1078 was modified such that an additional residue was added so that the loop region had the same number of residues as PT1079, however this modification failed to restore binding to PTmAb0005 or PTmAb0011. Thus indicating the importance of correct presentation of the peptides of the present invention to adopt a shape which closely resembles the native target in the context of the whole IgE molecule.

[image]

Summary

The work described herein shows that the monoclonal antibodies PTmAb0005 and PTmAb0011 specifically recognise P1. These antibodies have been used in phage display studies to identify mimotopes of the c-d loop of the Cε2 domain of IgE which are recognised by the monoclonal antibodies with high affinity.

7.5 Functional Characteristics of Characteristics of PTmAb0005 and PTmAb0011

The following experiments describe the functional characteristics of PTmAb0005 and PTmAb0011. Accordingly, the use of the targets of these antibodies will induce PTmAb0005 and PTmAb0011 like immune responses. The vaccination using these peptide based immunogens will, therefore, have the same functional characteristics.

### EXAMPLE 8

8.1 Materials and Methods

8.1.1 FcεRIα Binding Assay (Protein A Plates)

In this assay, a recombinant form of the ectodomain of the alpha chain of the high affinity receptor for IgE (alpha ectodomain) is utilised to bind chimaeric IgE. The carboxyl terminus of the alpha ectodomain is fused to a human IgG1 Fc sequence. This enables the recombinant molecule to be bound to protein A coated microtitre plates via the Fc region. Hence, the majority of the alpha ectodomain molecules should be available for binding ligand and provides a system for the analysis of IgE-receptor interactions. The format described below is aimed at detecting the (high affinity) receptor blocking activity of anti-IgE antibodies.

8.1.2 ELISA Protocol for Detection of Binding of IgE to the Alpha Chain Ectodomain of the High Affinity Receptor

Coat protein A plates with 100 μl/well α-ecto-Ig fusion protein diluted to 0.25 μg/ml in blocking buffer (PBS/5% BSA/0.05% Tween-20). Incubate 1 hour at 37° C. Dilute chimaeric IgE to 0.03125 μg/ml in 10% pig serum. Dilute anti-IgE antibody to appropriate test concentration(s) in this IgE solution. Incubate 1 hour at room temperature. Wash plates three times with PBS/0.05% Tween-20 using plate washer. Add 100 μl/well of IgE:anti-IgE solution (quadruplicates of each anti-IgE concentration are assayed). Incubate 1 hour at 37° C. Wash plates three times with PBS/0.05% Tween-20 using plate washer. Add 100 μl/well of goat anti-mouse lambda chain HRPO conjugated antibody diluted 1:6000 dilution in blocking buffer. Incubate 1 hour at 37° C. Wash plates three times with PBS/0.05% Tween-20 using plate washer. Add 200 μl/well of OPD substrate and incubate at room temperature in the dark for 2-10 minutes. Stop the reaction by the addition of 25 μl 25% H₂SO₄. Mix stopped reactions on plateshaker—SLOW speed. Read OD at 490 nm.

A figure for the percentage of inhibition of binding of IgE to its receptor can be calculated. A maximum binding value for IgE is determined from the average of a set of wells that contained IgE in 10% pig serum alone (i.e no anti-IgE). The % inhibition value is calculated thus:
(max IgE value−average of anti-IgE replicates)/max IgE value×100
8.1.3 FcεRIα Binding Assay (Clipped Ectodomain)

This assay is essentially identical to the previous assay except that the FcεRIα ectodomain/IgG construct is treated with the proteolytic enzyme Factor X to cleave the two moieties. The IgG Fc moiety is removed using protein A beads, and the Factor X is removed using strepatavidin beads, thus leaving an essentially pure alpha chain ectodomain product. In this assay format, the alpha ectodomain is bound directly to plastic microtitre plates, all other assay details are as described above.

8.1.4 CD23-Binding Assay (FcεRI, Low Affinity Receptor).

This assay was performed on either RPMI 8866 cells or primary human B-cells; two formats may be used, one for the detection of mAbs that bind to IgE associated with FcεRII, and a second that analysed whether the mAbs interfered with IgE association with FcεRII. For the first assay cells were loaded with chimaeric IgE (1 μg/ml) for an hour on ice in PBS, 1% FBS, 0.1% NaN₃. Excess IgE was removed and anti-IgE mAb added. Bound mAb was elucidated with FITC-conjugated rat anti-mouse IgG, antibody. For the second assay, chimaeric IgE (1 μg/ml) was pre-incubated with anti-IgE mAb for an hour at room temperature, with gentle mixing, prior to addition to the cells. The mixture was incubated with the cells for an hour on ice and then washed to remove unbound IgE. Bound IgE was detected with FITC-goat anti-human IgE or bound anti-IgE mAb was detected with FITC-conjugated rat anti-mouse IgG₁ antibody. Where studies were performed on PBMCs, constituent B-cells were identified with a PE-conjugated anti-CD19 antibody. Samples were analysed by flow cytometry.

8.2 Results

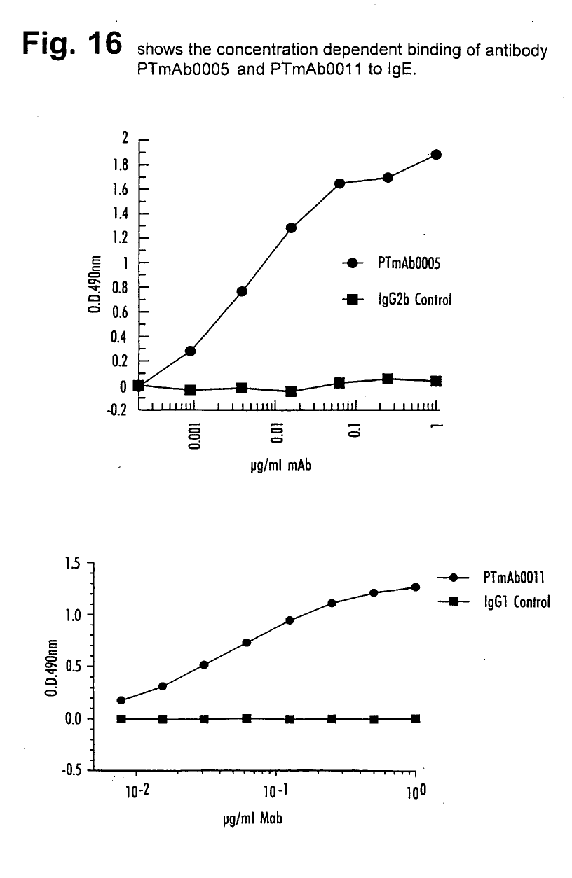

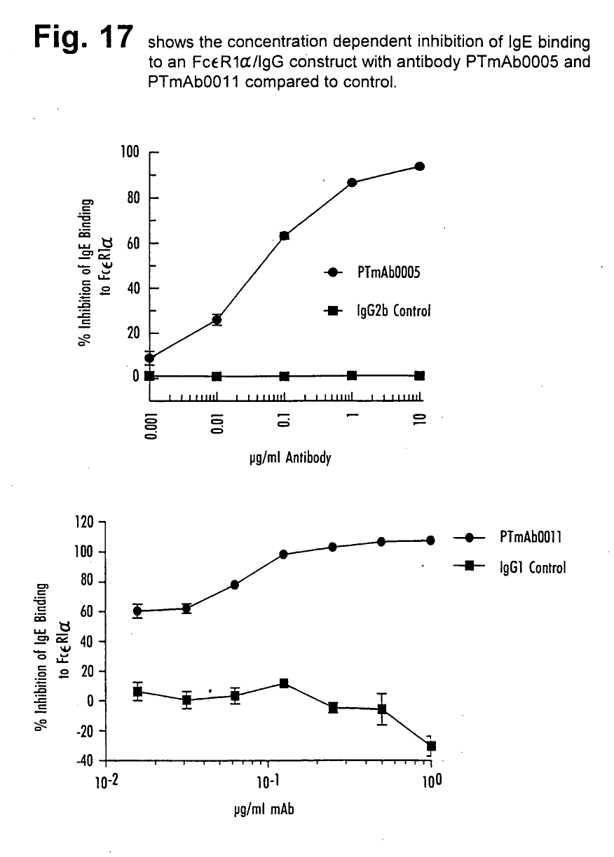

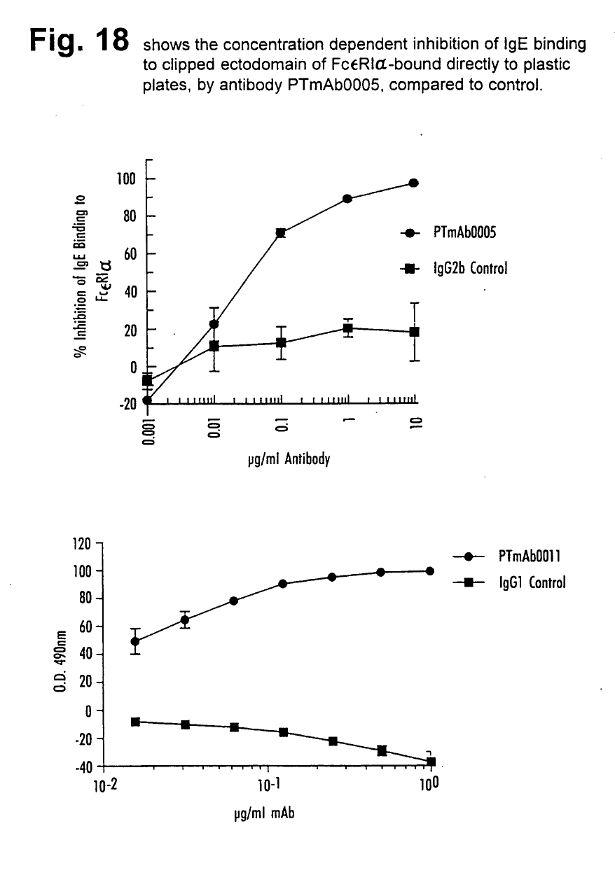

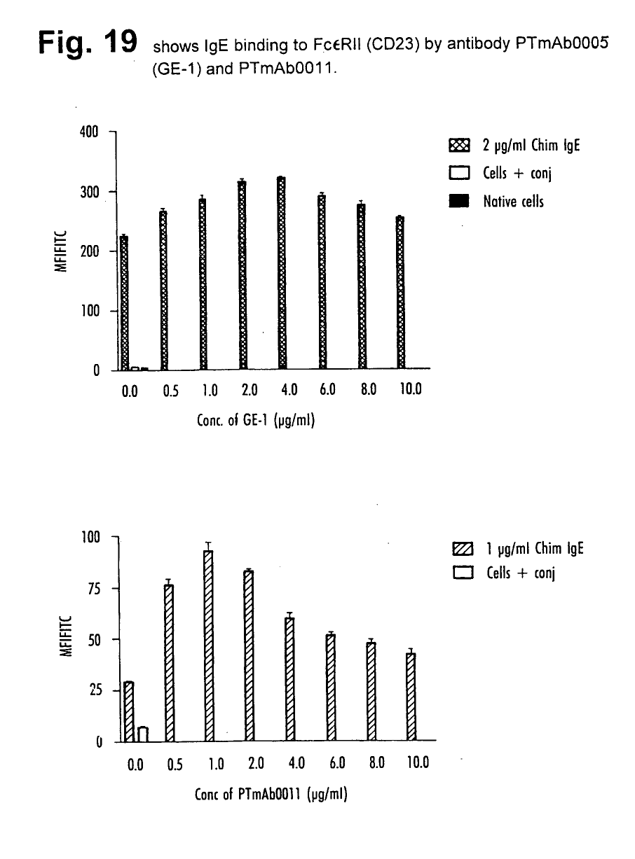

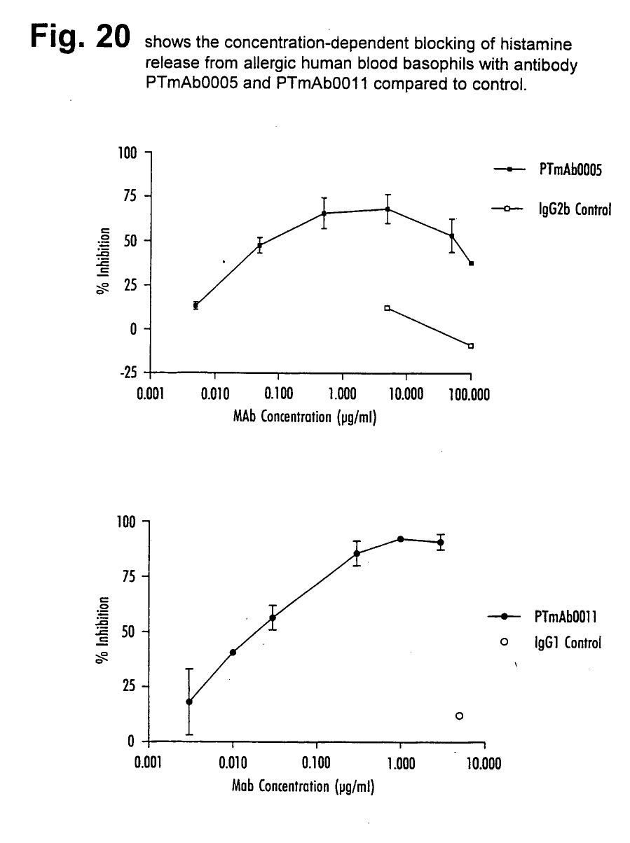

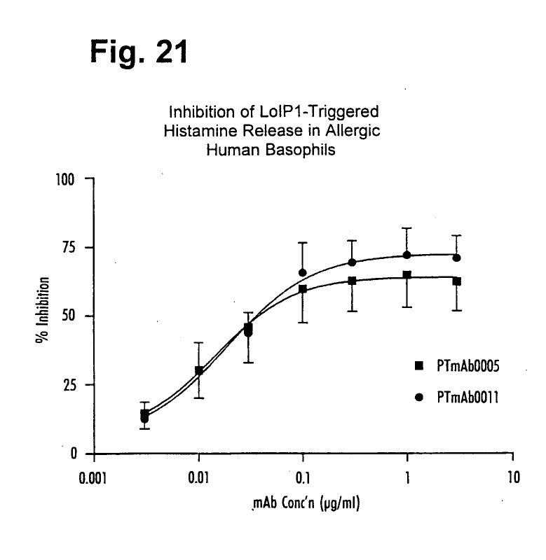

The results for both PTmAb0005 and PTmAb0011 are shown in FIGS. 16 to 21. FIG. 16 shows the concentration dependent binding of monoclonal antibody to plate bound IgE. FIG. 17 shows the concentration dependent inhibition of IgE binding to an FcεRIα/IgG construct with PTmAb0005 and PTmAb0011. FIG. 18 shows the inhibition of IgE binding to clipped ectodomain of FcεRIα-bound directly to plastic plates, by antibody PTmAb0005 and PTmAb0011. FIG. 19 shows the lack of inhibition of IgE binding to FcεRII (CD23) by antibody PTmAb0005 (clone GE-1) and PTmAb0011. FIG. 20 and 21, shows the concentration-dependent blocking of histamine release from allergic human blood basophils with antibody PTmAb0005 and PTmAb0011.

PTmAb0011 is a mouse monoclonal antibody with specificity for human IgE, showing no cross-reactivity with other human Ig isotypes or rat/mouse IgE. PTmAb0011 binds to both native and heat-treated IgE, when bound to an ELISA plate in a random orientation, indicating that its recognition site on IgE is not heat labile. PTmAb0011 also recognises IgE when bound via antigen to the ELISA plate. Importantly this mAb can completely block the interaction between human IgE and the α-chain binding component of the high affinity IgE receptor (FcεRI). However, this mAb still recognises human IgE when pre-bound to FcεRI, indicating that the mAb binding site is not lost upon receptor binding.

### EXAMPLE 9

9.1 Analysis of IgE Binding Properties of PTmAb0011 by Normal and Antigen Orientated ELISA

As described in Example 1, the normal IgE binding ELISA method was performed by coating plates with human chimaeric IgE, myeloma IgE, human Ig isotypes or rodent IgE (1 μg/ml in pH 9.6 carbonate/bicarbonate coating buffer). For antigen orientated ELISAs, NP-BSA was coated at a saturating concentration prior to the addition of chimaeric IgE (1 μg/ml). Alternatively, soluble human FcεRIα-chain was coated (0.25 μg/ml) followed by chimaeric IgE. The remaining ELISA was carried out as described in Experiment 1 (ELISA protocol for the detection of mouse anti-human IgE mAbs).

9.2 Results

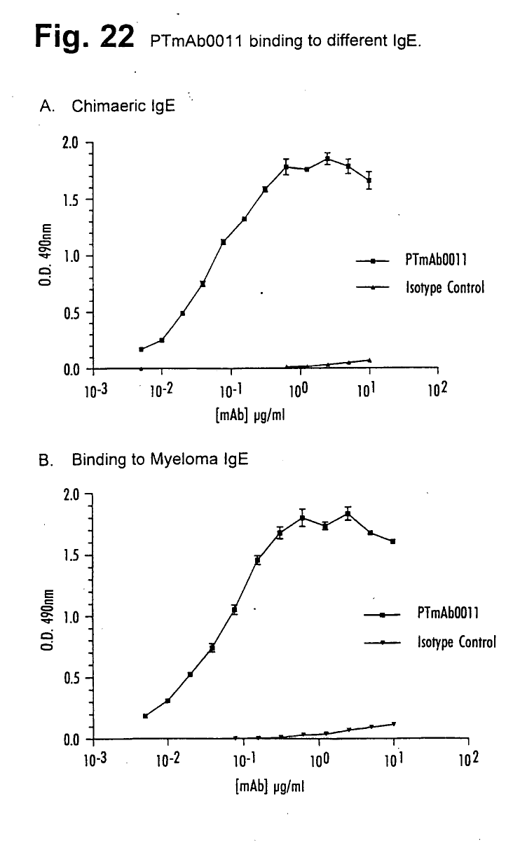

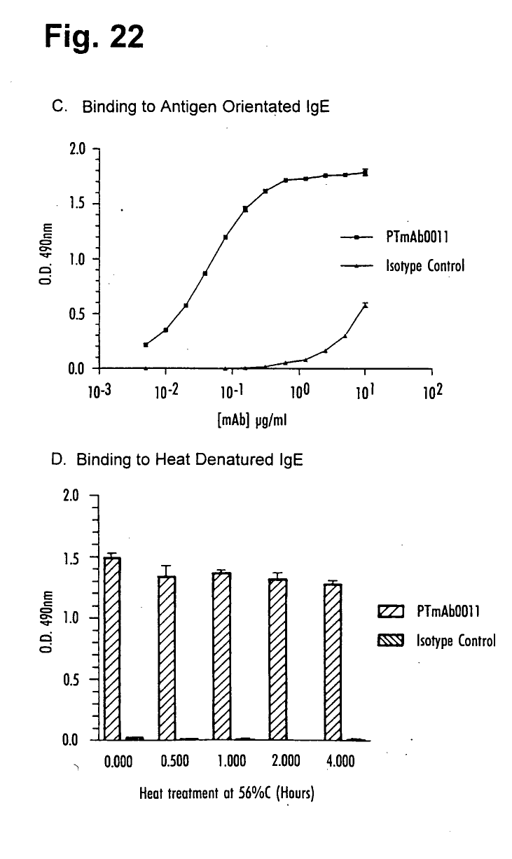

FIG. 22 illustrates that PTmAb0011 binds to both human/mouse chimaeric IgE and human myeloma IgE when bound to an ELISA plate in a random orientated manner. Similarly, binding to antigen orientated IgE (i.e IgE bound to plate bound NP-BSA) is dose dependent. PTmAb0011 was also analysed for its ability to recognise chimaeric IgE following heat treatment at 56° C. for a range of time periods. FIG. 22 also shows that the binding capacity of PTmAb0011 for IgE is unaffected by heat treatment.

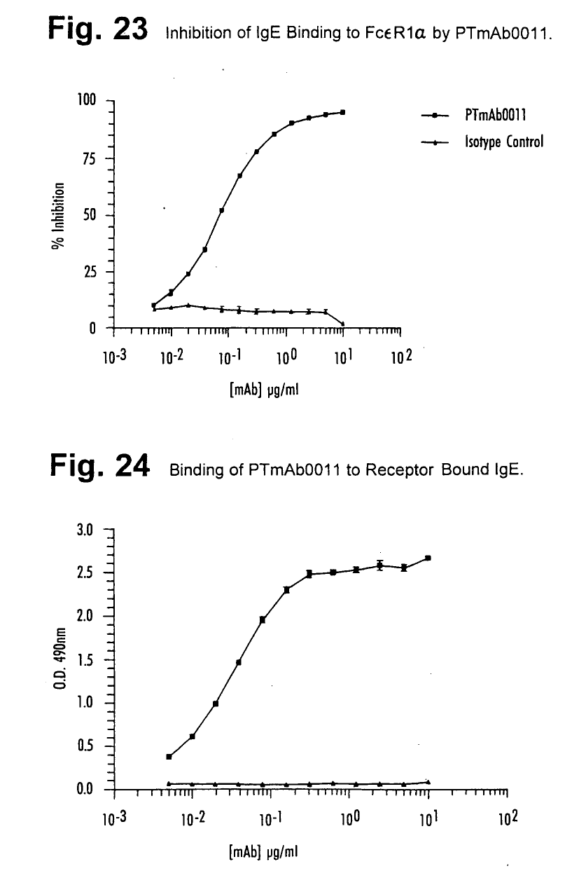

The mAb characterisation was further extended to determine whether PTmAb0011 was able to inhibit the interaction of IgE with the α-chain component of the high affinity IgE receptor (FIG. 23). Pre-incubation of IgE with PTmAb0011 prior to addition to plate bound FcεRI α-chain, resulted in a dose dependent inhibition of the interaction of IgE with FcεRI Ε-chain. PTmAb0011 was also (FIG. 24) recognises FcεRI α-chain associated IgE in a dose dependent manner.

### EXAMPLE 10

10.1 Analysis of IgE Secretion from Primary Human B-Cells

PBMCs were plated at 2×10⁵ cells per well in 96 U-well plates, in medium supplemented with both IL4 and anti-CD40. PTmAb0011 or isotype matched control mAb was added and cells incubated for 14 days prior to harvesting of supernatants for IgE analysis. Total IgE levels were measured by coating ELISA plates with rabbit anti-human IgE antibody (10 μg/ml) in 0.5M carbonate/bicarbonate buffer (pH9.6). Washed plates were blocked with PBS, 0.05% Tween 20, 5% BSA. Both cell supematants and IgE standard were incubated with saturating amounts of PTmAb0011 (10 μg/ml) for an hour at room temperature prior to addition to the ELISA plate to allow for IgE/anti-IgE complexes to be formed. Following incubation and washing steps, bound IgE was detected with HRP-sheep anti-human IgE, followed by OPD substrate. Levels of IgE in the cell supernatants were then estimated relative to the standard curve.

10.2 Results

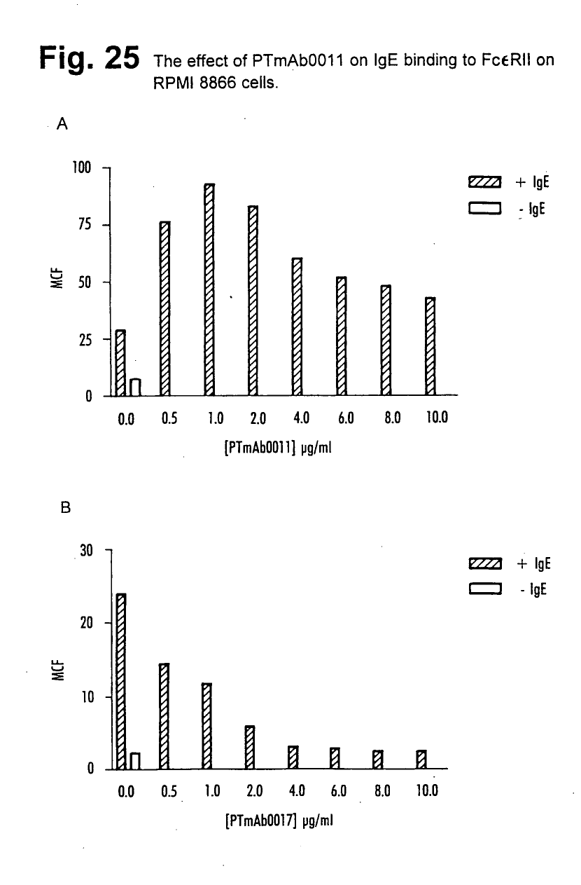

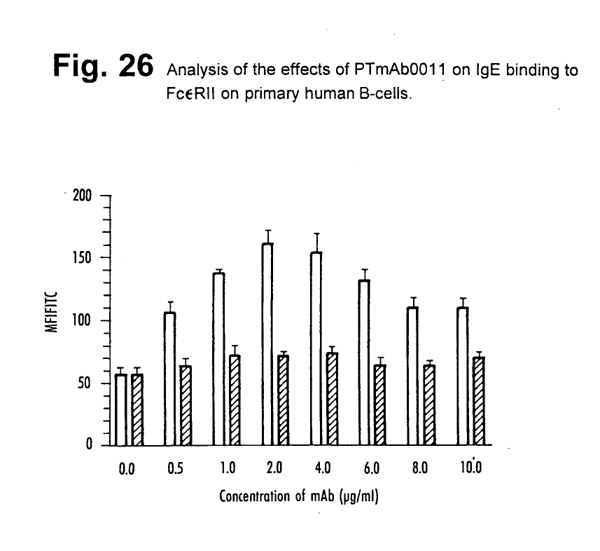

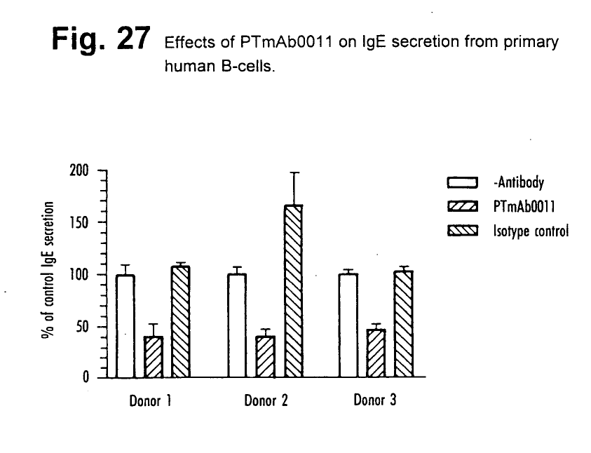

IgE was pre-incubated with PTmAb0011 over a dose range from 10 μg/ml to 0.5 μg/ml and examined for its effect on subsequent IgE binding to FcεRII on the human B-cell line RPM18866. FIG. 25 illustrates that pre-incubation of IgE with PTmAb0011 enhances IgE binding to FcεRII. A non P1 specific monoclonal antibody (PTmAb0017) did not enhance the binding of IgE to the FcεRII receptor. PTmAb0011 also enhances IgE binding to FcεRII on primary B-cells (FIG. 26).

10.3 Effects of PTmAb0011 on IgE Secretion from Primary Human B-Cells

Peripheral blood mononuclear cells were cultured with PTmAb0011, in the presence of additional IL-4 and anti-CD40 antibody to promote B-cell isotype switch to IgE secretion. An ELISA assay was developed that allowed for measurement of total IgE levels, that is free IgE and PTmAb0011 complexed IgE. To achieve such quantitation secreted IgE was pre-incubated with saturating levels of PTmAb0011 to allow for all of the IgE to be complexed. The total IgE within the tissue culture supernatant was quantitated relative to a standard curve of IgE that had also been complexed with saturating levels of PTmAb0011 . FIG. 27, illustrates that in three different donors, incubation of primary B-cells with PTmAb0011 (1 μg/ml) resulted in a significant reduction in the total levels of secreted IgE. No such inhibition was seen with the isotype matched control antibody.

10.4 Determination of Histamine Release from Human Basophils

Two assay formats were adopted. PBMC from non-allergic donors were passively sensitised with 1 μg/ml chimeric IgE for 30 min at 37° C., washed and treated with monoclonal antibodies for 30 min at 37° C. Alternatively PBMC from LolP1-sensitive donors were treated directly with monoclonal antibodies for 30 min at 37° C. Reactions were terminated by centrifugation. Histamine release in cell supernatants was determined by specific immunoassay (Immunotech 2562). Total cellular histamine content was determined in cells lysed with 0.5% Igepal detergent.

10.5 Basophil Blocking Assay

The ability of anti-IgE antibodies to block binding of chimeric IgE to FcεRI on human basophils was determined by incubation of PBMC from non-allergic donors with chimeric IgE in the presence of monoclonal antibodies and IL-3 for 30 min at 37° C. Cells were washed and histamine release was triggered with NP-BSA for a further 30 min at 37° C. Reactions were terminated by centrifugation and histamine release measured as above.

10.6 Allergic Basophil Inhibition Assay

The ability of anti-IgE antibodies to inhibit allergen-triggered degranulation was investigated by pre-incubating PBMC from LolP1-sensitive donors with monoclonal antibodies for 30 min at 37° C. prior to triggering with LolP1.

10. 7 Determination of Tryptase Release from Human Lung Mast Cells

Crude mast cell suspensions were prepared from human lung tissue by enzymatic digestion with a cocktail comprising hyaluronidase, pronase and DNAse. Cells were either used directly or pre-sensitised with chimeric IgE prior to treatment with anti-IgE antibodies. Mast cell degranulation was determined by colorimetric assay of the granule enzyme tryptase.

10.8 Determination of β-Hexosaminidase Release from RBL Cells Transfected with Human FcεR1α

Transfected cell line RBL J41 was obtained from Dr B. Helm, University of Sheffield. Cells were passively sensitised with either mouse monoclonal IgE anti-DNP or human chimeric IgE anti-NP and triggered with anti-human IgE antibodies. Degranulation was measured by the calorimetric assay of β-hexosaminidase release.

10.9 Results

10.9.1 Anaphylactogenicity of Anti-IgE Monoclonal Antibodies in Human Basophils

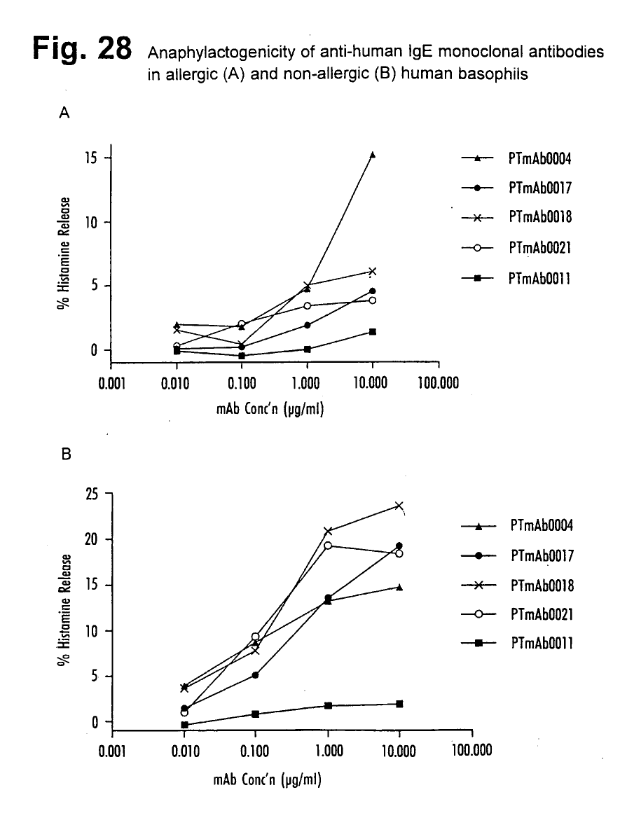

A number of different anti-IgE monoclonal antibodies were assayed for their ability to trigger histamine release from both allergic and non-allergic basophils (FIG. 28). In contrast to the other antibodies, PTmAb0011 was consistently unable to generate lo significant histamine release.

10.9.2 Anaphylactogenicity of Anti-IgE Monoclonal Antibodies in Human Lung Mast Cells

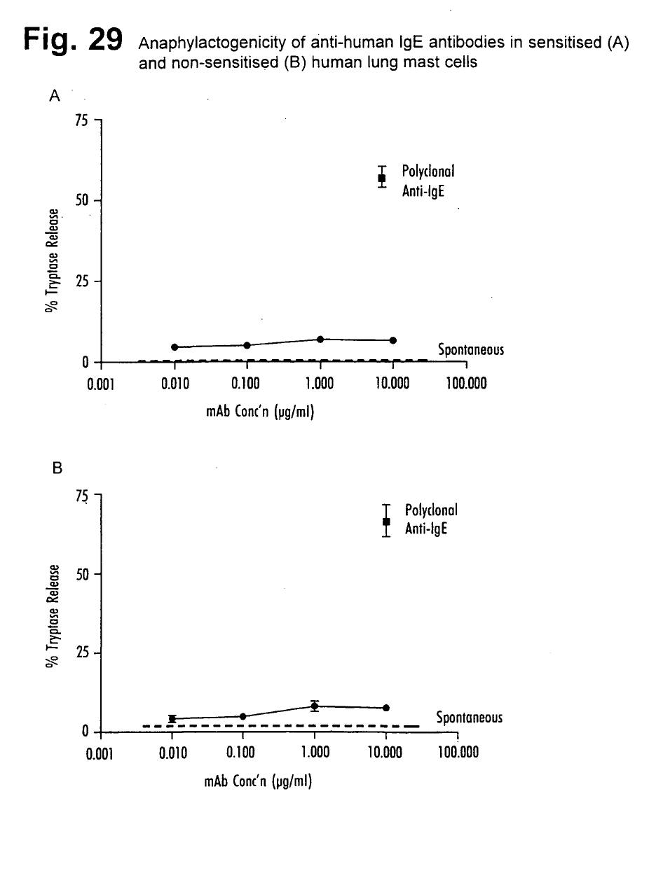

PTmAb0011 was also unable to release significant amounts of tryptase in both is sensitised and non-sensitised human lung mast cells (FIG. 29). Polyclonal anti-human IgE gave 60-70% release in these cells.

10.9.3 Anaphylactogenicity of Anti-IgE Monoclonal Antibodies in RBL Cells Transfected with Human FcεR1α

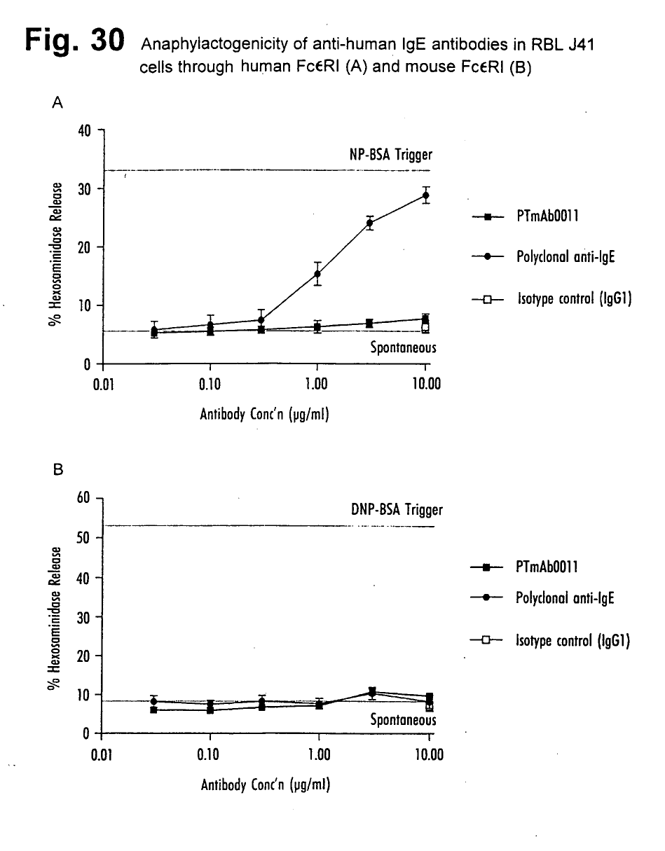

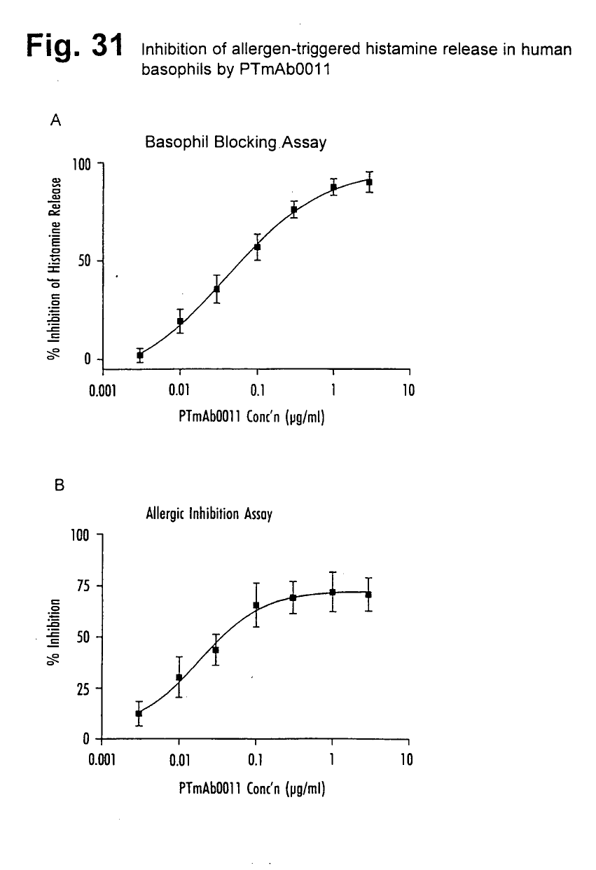

RBL J41 cells, passively sensitised with chimeric human IgE anti-NP, could be triggered with antigen NP-BSA and with polyclonal anti-human IgE but not with PTmAb0011 (FIG. 30). In contrast, when cells were sensitised with mouse IgE anti-DNP, both anti-human IgE antibodies were without effect. The cells could still be triggered by antigen DNP-BSA.

10.9.4 Basophil Blocking Assay

PTmAb0011 was able to block the binding of IgE to FcεR1 in non-allergic basophils and thus to inhibit subsequent triggering with NP-BSA antigen. The IC₅₀ value of this activity was around 60 ng/ml (FIG. 31). PTmAb0011 was also able to potently inhibit LolP1-triggered histamine release from allergic basophils with an IC₅₀ value of 40ng/ml (FIG. 31).

### EXAMPLE 11

### Monkey Passive Cutaneous Anapylalaxis Studies

PTmAb0005 and PTmAb0011 have also been tested for in vivo activity. Briefly, the local skin mast cells of African green monkeys were shaved and sensitised with intradermal administration of 100 ng of anti-NP IgE (human IgE anti-nitrophenylacetyl (NP) purchased from Serotech) into both arms. After 24 hours, a dose range of the monoclonal antibodies to be tested were injected at the same injection site as the human IgE on one arm. Control sites on the opposite arm of the same animals received either phosphate buffered saline (PBS) or non-specific human IgE (specific for Human Cytomegalovirus (CMV) or Human Immunodeficiency Virus (HIV)). After 5 hours, 10 mg of a BSA-NP conjugate (purchase from Biosearch Laboratories) was administered by intravenous injection. After 15-30 minutes, the control animals develop a readily observable roughly circular oedema from the anyphylaxis, which is measurable in millimeters. Results are expressed in either the mean oedema diameter of groups of three monkeys or as a percentage inhibition in comparison to PBS controls. Dec7B, is described in EP 0 477 231 B, which recognises a peptide 496-506 in the CF4 domain of human IgE, was used as a positive control.

| Amount of | | | | | |
|---|---|---|---|---|---|
| sample to | Mean diameter of oedema (mm) | | | | |
| be tested | | | | IgE α-CMV | IgE α-HIV |
| (μg) | mAb0005 | mAb0011 | Dec7B | control | control |
| 20 | 0 | 0 | 0 | 19.5 | 21 |
| 10 | 0 | 0 | 20 | 20.7 | 22 |
| 1 | 4 | 4.5 | 25 | 22.7 | 23 |
| 0.1 | 14.8 | 15.7 | 20 | 21.8 | 22.5 |
| 0.05 | 17.8 | 18.7 | 22.5 | 21.5 | 22.8 |
| PBS | 23.2 | 28.2 | 26 | 24.5 | 22.5 |

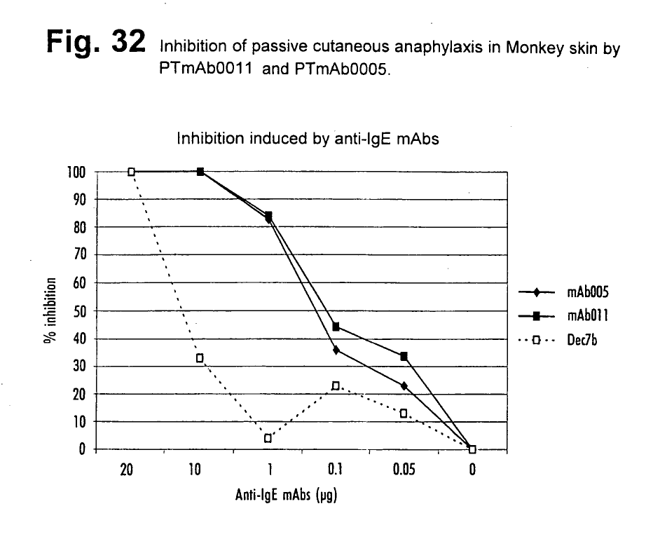

The percentage inhibtion of anaphylaxis are shown in FIG. 32.

TABLE 6

PTmAb0005 peptide ligands identified from primary
phage display biopannings with affinities
Human IgE Cε2 EDGQVMDVD - SEQ ID NO.1

| | | |
|---|---|---|
| Rank (ECL) | PTmAB BIAcore K_{D}^{Rel} (μM) | SEQ |

| | | | | | |
|---|---|---|---|---|---|
| Name | Library | PTmAb0005 | PTmAb0005 | PTmAb0011 | ID NO. |

| | | | | | | |
|---|---|---|---|---|---|---|
| IgEC67 | XCX15 | CFMNKQLADLELCPRE | 243 | 0.1 | 4.8 | 13 |
| IgEC26 | XCX15 | QCNAVLEGLQMVDHCWN; | 43 | 3 | 53.4 | 67 |
| IgEC29 | XCX15 | CCVADPETQMTPSSEMF; | 40 | >600 | >600 | 68 |
| IgEC42 | XCX15 | ECLKIEQQCADIVEIPR; | 15 | 19.4 | >500 | 69 |
| IgEC69 | XCX15 | SCAYTAQRQCSDVPNPG; | 11 | 6.6 | 6.4 | 70 |
| IgEC9 | XCX15 | ECRGPNMQMQDHCPTTD; | 10 | — | — | 71 |
| IgEC13 | XCX15 | ECLVYGQMADCAAGGWP; | 5 | >1000 | >1000 | 72 |
| IgEC56 | XCX15 | QCRQFVMNQSEKEFGQC; | 0 | 60 | >1000 | 73 |
| IgEC43 | XCX15 | HCKNEFKKGQWTYSCSD; | 0 | — | — | 74 |
| IgEC81 | XCX15 | CCVTDVQTTNMDVPAGQ; | 0 | 78 | 6.3 | 75 |
| IgEC83 | XCX15 | TCCVTDIPPPDYEQSLG; | 0 | — | — | 76 |
| IgEC70 | XCX15 | CCESDIPLNELHALADP; | 0 | — | — | 77 |
| IgEC64 | XCX15 | CCKSDIPSPVTQFNTMK; | 0 | — | — | 78 |
| IgEC73 | XCX15 | CCQSDVPHQPGINDLHV; | 0 | >600 | >600 | 79 |
| IgEC72 | XGX15 | CCMSDTPDISRLPVPDS; | 0 | — | — | 80 |
| IgEC66 | XCX15 | CCMSDSPADPNRGLPIW; | 0 | >600 | >600 | 71 |
| IgEC75 | XCX15 | CCLSDDAPTLPVRR; | 0 | — | — | 82 |
| ESC18 | XCX15 | CCITDVPQGVMYKGSPD; | 0 | — | — | 83 |
| ESC45 | XCX15 | ECKVDGQLSDSPLLRNN; | 0 | — | — | 84 |
| ESC12 | XCX15 | CCMTDDPMDPNSTWAIR; | 0 | — | — | 85 |
| ESC43 | XCX15 | CCMTDDPMYTNSTWAIR; | 0 | — | — | 86 |
| ESC1 | XCX15 | CCVDDTPNSGLAMRVSK; | 0 | — | — | 87 |
| ESC4 | XCX15 | CCEVDDFPTHHPGWTLR; | 0 | — | — | 88 |
| ESC46 | XCX15 | SCNLNHQSCDIPPVKQI; | 0 | — | — | 89 |
| ESC20 | XGX15 | CCMADQELDLGHNAANA; | 0 | — | — | 90 |
| | | | | | | 91 |
| ESD36 | XCX10 | CCVMDLELASGF; | 0 | — | — | 92 |
| ESD14 | XCX10 | CCVMDIEVRGSA; | 0 | — | — | 93 |
| ESD38 | XCX10 | CCQRDVELVFGS; | 0 | — | — | 94 |
| ESD15 | XCX10 | CCRADFEVGNGG; | 0 | — | — | 95 |
| ESD6/10/40 | XCX10 | CCVSDEPAGVRD; | 0 | — | — | 96 |
| ESB4/35 | XAX10 | GAGWQEKDKELR; | 0 | 70 | 700 | 96 |
| ESB25 | XAX10 | GAMTAGQLSDLP; | 0 | 60 | >1000 | 97 |
| ESB10/38 | XAX10 | VAGGQVVDRELK; | 0 | 139 | >1000 | 98 |
| ESB8 | XAX10 | KAGEQAMDMELR; | 0 | 257 | >1000 | 99 |
| ESB29/36 | XAX10 | RGRNQIMDLEI; | 0 | — | — | 100 |
| ESB15 | XAX10 | QIDRQITDTLL; | 0 | — | — | 101 |
| ESB26 | XAX10 | REQQISDVPRV; | 0 | — | — | 102 |
| ESB12 | XAX10 | CQAMDAEILNQV; | 0 | — | — | 103 |
| ESB1/6etc | XAX 10 | GQMMDTELLNR; | 0 | — | — | 104 |
| ESB7 | XAX10 | SMEGQVRDIQV; | 0 | — | — | 105 |
| ESB18 | XAX10 | YQQRDLELLAE; | 0 | — | — | 106 |
| ESB9 | XAX10 | SMGQKVDRELV; | 0 | — | — | 107 |
| ESB40 | XAX10 | SMGQEVDRELV; | 0 | — | — | 108 |
| SB21/33/31 | XAX10 | AENDQMVDWEI; | 0 | — | — | 109 |
| ESB32 | XAX10 | GGWQESDIPGR; | 0 | — | — | 110 |
| ESB4/35 | XAX10 | GGWQEKDKELR; | 0 | — | — | 111 |
| ESB24 | XAX10 | HCCRIDREVSGA; | 0 | — | — | 112 |
| ESB13 | XAX10 | CAPGMGCWESVK; | 0 | — | — | 113 |

TABLE 7

PTmAb0011 peptide ligands identified from primary
phage display biopannings with affinities
Human IgE Cε2 EDGQVMDVD - (SEQ ID NO.1)

| | | |
|---|---|---|
| Rank (ECL) | PTmAb BIAcore K_{D}^{Rel} (μM) | SEQ |

| | | | | | |
|---|---|---|---|---|---|
| Name | Library | PTmAb0011 | PTmAb0011 | PTmAb0005 | ID NO. |

| | | | | | | |
|---|---|---|---|---|---|---|
| EEC39/50/129 | XCX15 | SCREVWLGGSEMIMDCE; | 1611 | 2.4 | >1000 | 114 |
| EEC131 | XCX15 | SCPAFPREGDLCAPPTV; | 910 | 42 | >1000 | 115 |
| EEC147 | XCX15 | FCPEPICSPPLSRMTLS; | 883 | — | — | 116 |
| EEC40 | XCX15 | ECNQNLSGSLRHVDLNC; | 547 | — | — | 117 |
| EEC115/3/48 | XCX15 | RCDQQLPRDSYTFCMMS; | 438 | — | — | 118 |
| EEC36 | XCX15 | HCQQVFFPQDYLWCQRG; | 158 | — | — | 119 |
| EEC17/47/25 | XCX15 | DCEEPMCSPVLLQKLKP; | 147 | — | — | 120 |
| EEC40A | XCX15 | NCQDQMLREDAGCWSKI; | 80 | — | — | 121 |
| EEC51/48/53 | XCX15 | HCEEPEYSPATRVFCGR; | 75 | — | — | 122 |
| EEC2/23/44/132 | XCX15 | DCDWINPPDPPHFWKDT; | 33 | 7 | >600 | 123 |
| EEC41 | XCX15 | ACFSRNGQVTDVPHSCY; | 31 | — | — | 124 |
| EEC135 | XCX15 | KCPTYPKPNDRCLWPVP; | 19 | — | — | 125 |
| EEC116 | XCX15 | YCPKYPLEGDCLLDNDY; | 4 | — | — | 126 |
| EEC21/19 | XCX15 | RCEEWLCIPPAPAFAPP; | 3 | 27.8 | 14.9 | 127 |
| EEC55 | XCX15 | TCGQSELRCASLETHHV; | 0 | — | — | 128 |
| EEC5 | XCX15 | NCNDNPMLDCMPAWSS; | 0 | — | — | 129 |
| EEB33 | XAX10 | DALDERAWRARA; | 15 | 117 | >600 | 130 |
| EED183 | XCX10 | SCQGREVRRECW; | 596 | — | — | 131 |
| EED35/53/164 | XCX10 | VCDECVSRELAL; | 330 | — | — | 132 |
| EED38 | XCX10 | WCLEPECAPGLL; | 330 | — | — | 133 |
| EED147/173 | XCX10 | DCLSKGQMADLC; | 281 | — | — | 134 |
| EED35/153/164 | XCX10 | VCDECVSRELAL; | 118 | — | — | 135 |
| EED36 | XCX10 | GCPTWPRVGDHC; | 52 | — | — | 136 |
| EED131/138/102 | XCX10 | RCQSARVVPECW; | 32 | | — | 137 |
| EED18/47/48 | XCX10 | SCAPSGDCGYKG; | 31 | — | — | 138 |
| EED132 | XCX10 | GCPMWPQPDDEC; | 28 | — | — | 139 |
| EED139 | XCX10 | ECPRWPLMGDGC; | 26 | — | — | 140 |
| EED134 | XCX10 | GCQVGELVWCRE; | 14 | — | — | 141 |
| EED33 | XCX10 | QCVRDGTRKVCM; | 7 | — | — | 142 |
| EED50 | XCX10 | TCLVDRQESDVC; | 6 | — | — | 143 |
| EED34/104 | XCX10 | DCVVDGDRLVCL; | 3 | — | — | 144 |
| EED41/56 | XCX10 | RCEQGALRCVGE; | 0 | — | — | 145 |
| EED51 | XCX10 | VCPPGWKNLGCN; | 0 | — | — | 146 |
| EED57 | XCX10 | MCQGWEIVSECW; | 0 | — | — | 147 |

TABLE 8

IgEC67 mutants with improved affinity for PTmAb0005 and PTmAb0011
Mutants from the original sequence are shown in blue
Human IgE Cε2 EDGQVMDVD (SEQ ID NO.1)

| | | |
|---|---|---|
| | BIAcore K_{D}^{Rel} | |
| PTmAb Rank | (μM) | SEQ |

| | | | | | |
|---|---|---|---|---|---|
| Name | 0005 | 0011 | 0005 | 0011 | ID NO. |

| | | | | | | |
|---|---|---|---|---|---|---|
| IgEC67 | CFMNKQLADLLLCPRE | — | — | 0.1 | 4.8 | 13 |
| IgEG67-8 | CFINKQMADLELCPRE | 4.36 | 2.4 | 0.0094 | 0.066 | 12 |
| IgEC67-10 | ADGAGCFMNKQMADLELCPREAAEA; | 4.2 | 2.3 | — | — | 148 |
| IgEC67-1 | ADGAGCFMNKQMADLELCPRTAAEA; | 4.1 | 2.2 | — | — | 149 |
| IgEC67-2 | ADGAACFMNKQMADLELCPRVAAEA; | 3.2 | 1.8 | — | — | 150 |
| IgEC67-3 | ADGAGCFINKQLADLELCPRVAAEA; | 3 | 1.7 | — | — | 151 |
| IgEC67-12 | ADGAGCFINKQLADLELCPREAAEA; | 3 | 1.9 | — | — | 152 |
| IgEC67-9 | ADGAGCFMNKQLADLEMCPRDDAEA; | 2.7 | 2.4 | — | — | 153 |
| IgEC67-4 | ADGAGCFMNKQLADPELCPREAEEA; | 2.4 | 1.3 | — | — | 154 |
| IgEC67-5 | ADGAGCFMNKQLVDLELCPRGAAEA; | 1.9 | 1.6 | — | — | 155 |
| IgEC67-6 | ADGAGCFMNNQLADWELCPRAAAEA; | 1.9 | 1.6 | — | — | 156 |
| IgEC67-11 | ADGAGCFMNKQMADWEMCPRAAAEA; | 1.8 | 1.9 | — | — | 157 |
| IgEC67-14 | ADGAGCFMNKQQADLELCPRGAAEA; | 1.2 | 0.9 | — | — | 158 |
| IgEC67-13 | ADGAECFMNKQLADSELCPRVAAEA; | 1.1 | 0.8 | — | — | 159 |
| IgEC67-7 | ADGAGCFMNKQLADLELCPREAAEA; | 1 | 1 | — | — | 160 |

TABLE 9

IgEC67-8 Mutants with Improved Affinity for PtmAb0005
Human IgE Cs2 EDGQVMDVD (SEQ ID NO.1)

| | | | |
|---|---|---|---|
| Clone | Rel. Rank A | Rel. Rank B | SEQ ID NO. |

| | | | | |
|---|---|---|---|---|
| IgEC67-8 | GCFINKQMADLELCPRE | 1.00 | 1.00 | 12 |

Mutants with improved affinity for PTmAb0005

| | | | | |
|---|---|---|---|---|
| 1-3 | ADGAGCFINMQMADQELCPRAAAEA; | 1.73 | 1.31 | 161 |
| 2-13 | ADGAGCFINKQMSDFELCPREAGEA; | 1.56 | 2.14 | 162 |
| 3-11 | ADGAGCFINKQMADLELCTREAAEA; | 1.54 | 2.02 | 163 |
| 3-1,3-9,3-10 | ADGAGCFINKQMADLELCPRQAAEA; | 1.54 | 1.85 | 164 |
| 1-11 | ADGAGCFINNQMADLELCPRGGAEA; | 1.45 | 1.32 | 165 |
| 2-15 | ADGAGCFINKQMADWELCPREGAEA; | 1.44 | 1.57 | 166 |
| 4-9 | ADGAGCFINKQMADLELCPSQAAEA; | 1.38 | 1.70 | 167 |
| 1-4,1-2,1-12 | ADGAGCFINKQMADLELCPREGAEA; | 1.37 | 1.39 | 168 |
| 5-16 | ADGAGCFINKQMADSELCPREPAEA; | 1.29 | 1.83 | 169 |
| 4-1 | ADGAGCFIKKQMADLELCPREAWEA; | 1.24 | 1.52 | 170 |
| 2-12 | ADGAECFINKQMADRELCAREVAEA; | 1.22 | 1.50 | 171 |
| 1-9,2-5 | ADGAGCFIDKQMADLELCPRAAAEA; | 1.21 | 1.41 | 172 |
| 2-9,2-6 | ADGAGCFINKQMADLELCRREAGEA; | 1.19 | 1.54 | 173 |
| 1-16 | ADGAGCFKNKQMVDSELCARQAAEA; | 1.14 | 1.17 | 174 |
| 1-5 | ADGAGCFQNKQMADLELCPREAAEA; | 1.13 | 1.73 | 175 |
| 4-2,4-3 | ADGAECFINKQRADLELCPGEAAEA; | 1.11 | 1.60 | 176 |
| 1-10 | ADGAGCFINKQMADSELCPAAAAEA; | 1.10 | 1.08 | 177 |

Mutants with similar affinity for PTmAB0005

| | | | | |
|---|---|---|---|---|
| 5-11 | ADGAGCFINRQMADPELCPREAAEA; | 1.09 | 1.97 | 178 |
| 1-8 | ADGAGCFIEKQMADMELCQARAAEA; | 1.08 | 1.32 | 179 |
| 5-10 | ADGAGCFINKQMADWELCPREAAEA; | 1.05 | 1.83 | 180 |
| 5-2 | ADGAGCFINNQMADLELCPREAAEA; | 1.04 | 1.24 | 181 |
| 1-1 | ADGAGCFIEKQMADMELCQRETAEA; | 1.04 | 1.29 | 182 |
| 2-3 | ADGAGCFINKQMADMELCPREAAEA; | 1.03 | 1.31 | 183 |
| 2-8,1-13,4-11,1-14 | ADGAGCFINKQMADLELCPREAAEA; | 1.00 | 1.00 | 184 |
| 1-6 | ADGAGCFRNKQMADLELCPREAAEA; | 0.95 | 1.16 | 185 |
| 1-7 | ADGAGCFINKQMADLELCPARAAEA; | 0.91 | 1.25 | 186 |
| 2-11,2-4,2-10,2-7 | ADGAGCFINRQLADMELCSRGAAEA; | 0.79 | 1.39 | 187 |
| 4-4 | ADGAECFINRQMADLELCGREAAEA; | 0.69 | 1.03 | 188 |

Mutants with affinity for streptavidin

| | | | | |
|---|---|---|---|---|
| 6-9,5-1,6-2,6-8,6-4 | ADGAGCFISPQLADWKRCMREAAEA; | 1.53 | 1.19 | 189 |
| 6-12,5-8 | ADGAGCSIHTQMADWERCLREGAEA; | 0.93 | 0.67 | 190 |
| 6-10 | ADGAGCSIHRQMADWERCLREGAEA; | 0.91 | 0.69 | 191 |

1-41. (canceled)

42. A peptide, or mimotope thereof of less than 100 amino acids in length, comprising an isolated surface exposed epitope of a Cε2 domain of IgE, wherein said surface exposed epitope of Cε2 is P1 (SEQ ID NO. 1).

43. A peptide as claimed in claim 42 wherein the surface exposed epitope of Cε2 is P2 (SEQ ID NO. 2), or a mimotope thereof.

44. A peptide as claimed in claim 42 wherein the surface exposed epitope of Cε2 is P3 (SEQ ID NO. 3), or a mimotope thereof.

45. A peptide as claimed in claim 42 wherein the surface exposed epitope of Cε2 is P4 (SEQ ID NO. 4), or a mimotope thereof.

46. A peptide as claimed in claim 42 wherein the surface exposed epitope of Cε2 is P5 (SEQ ID NO. 5), or mimotope thereof.

47. A peptide as claimed in claim 42 wherein the surface exposed epitope of Cε2 is P6 (SEQ ID NO. 6), or a mimotope thereof.

48. A peptide as claimed in claim 42 wherein the surface exposed epitope of Cε2 is P7 (SEQ ID NO. 7), or a mimotope thereof.

49. A mimotope as claimed in claim 42 wherein the mimotope is a peptide.

50. The peptide, or mimotope thereof as claimed in claim 42 wherein the isolated epitope is derived from a loop structure of the Cε2 domain of IgE.

51. The peptide, or mimotope thereof as claimed in claim 51, wherein the loop structure of the CP2 domain of IgE is a A-B or a C-D loop.

52. A peptide as claimed in claim 43 wherein the mimotope of P1 is a peptide of the general formula: h x d h h a n a n x y;
wherein: h is a hydrophobic amino acid residue; d is an ionic bond donating amino acid residue; a is an acidic amino acid residue; n is an ionically neutral/non-polar amino acid residue; and x is an amino acid.

53. A peptide as claimed in claim 43, wherein the mimotope of P1 is a peptide of the general formula:
Q, X₁, M, D, X₁, X₂, X₃
wherein X₁ is selected from V, I, L, M, F or A; X₂ is selected from D or E; and X₃ is selected from L, I, V, M, A or F.

54. A peptide as claimed in claim 43 wherein the mimotope of P1 is selected from the group consisting of P15q (SEQ ID NO. 11), PT1079 (SEQ ID NO. 13), PT1079GS (SEQ ID NO. 15), PT1078 (SEQ ID NO. 16), and PT15 (SEQ ID NO. 8).

55. A peptide as claimed in claim 44, wherein the mimotope of P2 is P16 (SEQ ID NO. 24).

56. A peptide as claimed in claim 45 wherein the mimotope of P3 is P17 (SEQ ID NO. 26).

57. An immunogenic composition for the treatment of allergy comprising the peptide, or mimotope thereof as claimed in claim 42, additionally comprising a carrier molecule.

58. The immunogenic composition as claimed in claim 58, wherein the carrier molecule is selected from Protein D or Hepatitis B core antigen.

59. An immunogenic composition for the treatment of allergy comprising the peptide, or mimotope thereof as claimed in claim 42, wherein the immunogenic composition is a chemical conjugate of the peptide or mimotope thereof.

60. The immunogenic composition as claimed in claim 58, wherein the peptide, or mimotope thereof is presented within the primary sequence of the carrier.

61. The immunogenic composition as claimed in claim 60, wherein the peptide, or mimotope thereof is presented within the primary sequence of the carrier.

62. A vaccine for the treatment of allergy comprising the immunogenic composition as claimed in claim 58, further comprising an adjuvant.

63. A vaccine for the treatment of allergy comprising the immunogenic composition as claimed in claim 59, further comprising an adjuvant.

64. A vaccine for the treatment of allergy comprising the immunogenic composition as claimed in claim 60, further comprising an adjuvant.

65. A vaccine for the treatment of allergy comprising the immunogenic composition as claimed in claim 61, further comprising an adjuvant.

66. A vaccine for the treatment of allergy comprising the immunogenic composition as claimed in claim 62, further comprising an adjuvant.

67. A ligand which is capable of recognizing a surface exposed epitope of the Cε2 domain of IgE, characterized in that the ligand is not PTmAb0005.

68. A ligand as claimed in claim 68, wherein the ligand is PTmAb0011 deposited under the Budapest Treaty patent deposit at ECACC on Mar. 8^{th}, 1999 under Accession No. 99030805.

69. A pharmaceutical composition comprising a ligand which is capable of recognizing a surface exposed epitope of the Cε2 domain of IgE.

70. A pharmaceutical composition as claimed in claim 70 wherein the ligand is capable of recognizing the C-D Loop of the Cε2 domain of IgE.

71. A pharmaceutical composition as claimed in claim 71, wherein the ligand is a monoclonal antibody selected from PTmAb0005 or PTmAb0011.

72. A peptide which is recognized by PTmAb0005 or PTmAb0011.

73. An immunogen comprising a peptide as claimed in claim 73.

74. A method of manufacturing a vaccine comprising the manufacture of an immunogen as claimed in claim 58, and formulating the immunogen with an adjuvant.

75. A method of manufacturing a vaccine comprising the manufacture of an immunogen as claimed in claim 60, and formulating the immunogen with an adjuvant.

76. A method for treating a patient suffering from or susceptible to allergy, comprising the administration of a peptide as claimed in any one of claims 42, to the patient.

77. A method for treating a patient suffering from or susceptible to allergy, comprising the administration of a vaccine as claimed in claim 63 to the patient.

78. A method for treating a patient suffering from or susceptible to allergy, comprising the administration of a vaccine as claimed in claim 64 to the patient.

79. A method for treating a patient suffering from or susceptible to allergy, comprising the administration of a vaccine as claimed in claim 65 to the patient.

80. A method for treating a patient suffering from or susceptible to allergy, comprising the administration of a vaccine as claimed in claim 66 to the patient.

81. A method for treating a patient suffering from or susceptible to allergy, comprising the administration of a vaccine as claimed in claim 67 to the patient.

82. A method of treating a patient suffering from or susceptible to allergy comprising administration of a pharmaceutical composition as claimed in any one of claims 65, to the patient.

83. An immunogenic composition for the treatment of allergy comprising the peptide, or mimotope thereof as claimed in claim 42, wherein the immunogenic composition is expressed as a fusion protein, and a carrier molecule.

84. The immunogenic composition as claimed in claim 84, wherein the peptide, or mimotope thereof is presented within the primary sequence of the carrier.

85. A vaccine for the treatment of allergy comprising the immunogenic composition as claimed in claim 84, further comprising an adjuvant.


SEQUENCE LISTING (193 sequences)

SEQ ID NO: 1 - protein, 9 aa, Artificial Sequence
  Chimeric
EDGQVMDVD

SEQ ID NO: 2 - protein, 8 aa, Artificial Sequence
  Chimeric
STTQEGEL

SEQ ID NO: 3 - protein, 10 aa, Artificial Sequence
  Chimeric
SQKHWLSDRT

SEQ ID NO: 4 - protein, 10 aa, Artificial Sequence
  Chimeric
GHTFEDSTKK

SEQ ID NO: 5 - protein, 8 aa, Artificial Sequence
  Chimeric
GGGHFPPT

SEQ ID NO: 6 - protein, 6 aa, Artificial Sequence
  Chimeric
PGTINI

SEQ ID NO: 7 - protein, 5 aa, Artificial Sequence
  Chimeric
FTPPT

SEQ ID NO: 8 - protein, 13 aa, Artificial Sequence
  Chimeric
CLEDGQVMDVDLL

SEQ ID NO: 9 - protein, 13 aa, Artificial Sequence
  Chimeric
LLDVDMVQGDELC

SEQ ID NO: 10 - protein, 13 aa, Artificial Sequence
  Chimeric
WLEDGQVMDVDLC

SEQ ID NO: 11 - protein, 13 aa, Artificial Sequence
  Chimeric
CLEDGQVMDVDLC

SEQ ID NO: 12 - protein, 16 aa, Artificial Sequence
  Chimeric
CFINKQMADLELCPRE

SEQ ID NO: 13 - protein, 16 aa, Artificial Sequence
  Chimeric
CFMNKQLADLELCPRE

SEQ ID NO: 14 - protein, 22 aa, Artificial Sequence
  Chimeric
CLEDGQVMDVDLCPREAAEGDK

SEQ ID NO: 15 - protein, 20 aa, Artificial Sequence
  Chimeric
CLEDGQVMDVDLCGGSSGGP

SEQ ID NO: 16 - protein, 21 aa, Artificial Sequence
  Chimeric
CLEDGQVMDVDCPREAAEGDK

SEQ ID NO: 17 - protein, 7 aa, Artificial Sequence
  Chimeric
QVMDVDL

SEQ ID NO: 18 - protein, 17 aa, Artificial Sequence
  Chimeric
KCREVWLGESETIMDCE

SEQ ID NO: 19 - protein, 17 aa, Artificial Sequence
  Chimeric
ACREVWLGESETIMDCD

SEQ ID NO: 20 - protein, 17 aa, Artificial Sequence
  Chimeric
SCREVWLGESETVMDCG

SEQ ID NO: 21 - protein, 17 aa, Artificial Sequence
  Chimeric
NCQDLMLREDAGCWSKM

SEQ ID NO: 22 - protein, 17 aa, Artificial Sequence
  Chimeric
DCEEPMCSPVLLQQLKL

SEQ ID NO: 23 - protein, 10 aa, Artificial Sequence
  Chimeric
LEDGQVMDVD

SEQ ID NO: 24 - protein, 10 aa, Artificial Sequence
  Chimeric
CSTTQEGELA

SEQ ID NO: 25 - protein, 6 aa, Artificial Sequence
  Chimeric
TTQEGE

SEQ ID NO: 26 - protein, 11 aa, Artificial Sequence
  Chimeric
CSQKHWLSDRT

SEQ ID NO: 27 - protein, 22 aa, Artificial Sequence
  Chimeric
TYQGHTFEDSTKKCADSNPRGV

SEQ ID NO: 28 - protein, 6 aa, Artificial Sequence
  Chimeric
GGHFPP

SEQ ID NO: 29 - protein, 13 aa, Artificial Sequence
  Chimeric
CFINKQMADLELC

SEQ ID NO: 30 - protein, 13 aa, Artificial Sequence
  Chimeric
CFMNKQLADLELC

SEQ ID NO: 31 - protein, 16 aa, Artificial Sequence
  Chimeric
KCREVWLGESETIMDC

SEQ ID NO: 32 - protein, 17 aa, Artificial Sequence
  Chimeric
HCQQVFFPQDYLWCQRG

SEQ ID NO: 33 - protein, 17 aa, Artificial Sequence
  Chimeric
SCREVWLGGSEMIMDCE

SEQ ID NO: 34 - protein, 17 aa, Artificial Sequence
  Chimeric
ECNQNLSGSLRHVDLNC

SEQ ID NO: 35 - protein, 17 aa, Artificial Sequence
  Chimeric
DCEEPMCSPVLLQKLKP

SEQ ID NO: 36 - protein, 17 aa, Artificial Sequence
  Chimeric
SCREVWLGGSEMIMDCE

SEQ ID NO: 37 - protein, 17 aa, Artificial Sequence
  Chimeric
RCDQQLPRDSYTFCMMS

SEQ ID NO: 38 - protein, 17 aa, Artificial Sequence
  Chimeric
SCPAFPREGDLCAPPTV

SEQ ID NO: 39 - protein, 17 aa, Artificial Sequence
  Chimeric
FCPEPICSPPLSRMTLS

SEQ ID NO: 40 - protein, 12 aa, Artificial Sequence
  Chimeric
VCDECVSRELAL

SEQ ID NO: 41 - protein, 12 aa, Artificial Sequence
  Chimeric
WCLEPECAPGLL

SEQ ID NO: 42 - protein, 12 aa, Artificial Sequence
  Chimeric
VCDECVSRELAL

SEQ ID NO: 43 - protein, 12 aa, Artificial Sequence
  Chimeric
DCLSKGQMADLC

SEQ ID NO: 44 - protein, 12 aa, Artificial Sequence
  Chimeric
SCQGREVRRECW

SEQ ID NO: 45 - protein, 17 aa, Artificial Sequence
  Chimeric
WCREVWLGESETIMDCE

SEQ ID NO: 46 - protein, 17 aa, Artificial Sequence
  Chimeric
ACREVWLGESETIMDCD

SEQ ID NO: 47 - protein, 17 aa, Artificial Sequence
  Chimeric
GCAEPKCWQALHQKLKP

SEQ ID NO: 48 - protein, 17 aa, Artificial Sequence
  Chimeric
ECRGPNMQMQDHCPTTD

SEQ ID NO: 49 - protein, 17 aa, Artificial Sequence
  Chimeric
QCNAVLEGLQMVDHCWN

SEQ ID NO: 50 - protein, 17 aa, Artificial Sequence
  Chimeric
CCVADPETQMTPSSEMF

SEQ ID NO: 51 - protein, 17 aa, Artificial Sequence
  Chimeric
HCKNEFKKGQWTYSCSD

SEQ ID NO: 52 - protein, 17 aa, Artificial Sequence
  Chimeric
QCRQFVMNQSEKEFGQC

SEQ ID NO: 53 - protein, 17 aa, Artificial Sequence
  Chimeric
NCFMNKQLADLELCPRE

SEQ ID NO: 54 - protein, 17 aa, Artificial Sequence
  Chimeric
SCAYTAQRQCSDVPNPG

SEQ ID NO: 55 - protein, 19 aa, Artificial Sequence
  Chimeric
GCFMNKQMADLELCPRTAA

SEQ ID NO: 56 - protein, 19 aa, Artificial Sequence
  Chimeric
ACFMNKQMADLELCPRVAA

SEQ ID NO: 57 - protein, 19 aa, Artificial Sequence
  Chimeric
GCFINKQLADLELCPRVAA

SEQ ID NO: 58 - protein, 19 aa, Artificial Sequence
  Chimeric
GCFMNKQLADWELCPRAAA

SEQ ID NO: 59 - protein, 19 aa, Artificial Sequence
  Chimeric
ECFMNKQLADSELCPRVAA

SEQ ID NO: 60 - protein, 19 aa, Artificial Sequence
  Chimeric
GCFMNKQLADPELCPREAE

SEQ ID NO: 61 - protein, 19 aa, Artificial Sequence
  Chimeric
GCFMNKQLVDLELCPRGAA

SEQ ID NO: 62 - protein, 19 aa, Artificial Sequence
  Chimeric
GCFMNKQLADLELCPREAA

SEQ ID NO: 63 - protein, 19 aa, Artificial Sequence
  Chimeric
GCFMNKQQADLELCPRGAA

SEQ ID NO: 64 - protein, 19 aa, Artificial Sequence
  Chimeric
GCFINKQMADLELCPREAA

SEQ ID NO: 65 - protein, 20 aa, Artificial Sequence
  Chimeric
CLEDGQVMDVDCPREAAEGD

SEQ ID NO: 66 - protein, 21 aa, Artificial Sequence
  Chimeric
CLEDGQVMDVDLCPREAAEGD

SEQ ID NO: 67 - protein, 17 aa, Artificial Sequence
  Chimeric
QCNAVLEGLQMVDHCWN

SEQ ID NO: 68 - protein, 17 aa, Artificial Sequence
  Chimeric
CCVADPETQMTPSSEMF

SEQ ID NO: 69 - protein, 17 aa, Artificial Sequence
  Chimeric
ECLKIEQQCADIVEIPR

SEQ ID NO: 70 - protein, 17 aa, Artificial Sequence
  Chimeric
SCAYTAQRQCSDVPNPG

SEQ ID NO: 71 - protein, 17 aa, Artificial Sequence
  Chimeric
ECRGPNMQMQDHCPTTD

SEQ ID NO: 72 - protein, 17 aa, Artificial Sequence
  Chimeric
ECLVYGQMADCAAGGWP

SEQ ID NO: 73 - protein, 17 aa, Artificial Sequence
  Chimeric
QCRQFVMNQSEKEFGQC

SEQ ID NO: 74 - protein, 17 aa, Artificial Sequence
  Chimeric
HCKNEFKKGQWTYSCSD

SEQ ID NO: 75 - protein, 17 aa, Artificial Sequence
  Chimeric
CCVTDVQTTNMDVPAGQ

SEQ ID NO: 76 - protein, 17 aa, Artificial Sequence
  Chimeric
TCCVTDIPPPDYEQSLG

SEQ ID NO: 77 - protein, 17 aa, Artificial Sequence
  Chimeric
CCESDIPLNELHALADP

SEQ ID NO: 78 - protein, 17 aa, Artificial Sequence
  Chimeric
CCKSDIPSPVTQFNTMK

SEQ ID NO: 79 - protein, 17 aa, Artificial Sequence
  Chimeric
CCQSDVPHQPGINDLHV

SEQ ID NO: 80 - protein, 17 aa, Artificial Sequence
  Chimeric
CCMSDTPDISRLPVPDS

SEQ ID NO: 81 - protein, 17 aa, Artificial Sequence
  Chimeric
CCMSDSPADPNRGLPIW

SEQ ID NO: 82 - protein, 14 aa, Artificial Sequence
  Chimeric
CCLSDDAPTLPVRR

SEQ ID NO: 83 - protein, 17 aa, Artificial Sequence
  Chimeric
CCITDVPQGVMYKGSPD

SEQ ID NO: 84 - protein, 17 aa, Artificial Sequence
  Chimeric
ECKVDGQLSDSPLLRNN

SEQ ID NO: 85 - protein, 17 aa, Artificial Sequence
  Chimeric
CCMTDDPMDPNSTWAIR

SEQ ID NO: 86 - protein, 17 aa, Artificial Sequence
  Chimeric
CCMTDDPMYTNSTWAIR

SEQ ID NO: 87 - protein, 17 aa, Artificial Sequence
  Chimeric
CCVDDTPNSGLAMRVSK

SEQ ID NO: 88 - protein, 17 aa, Artificial Sequence
  Chimeric
CCEVDDFPTHHPGWTLR

SEQ ID NO: 89 - protein, 17 aa, Artificial Sequence
  Chimeric
SCNLNHQSCDIPPVKQI

SEQ ID NO: 90 - protein, 17 aa, Artificial Sequence
  Chimeric
CCMADQELDLGHNAANA

SEQ ID NO: 91 - protein, 12 aa, Artificial Sequence
  Chimeric
CCVMDLELASGF

SEQ ID NO: 92 - protein, 12 aa, Artificial Sequence
  Chimeric
CCVMDIEVRGSA

SEQ ID NO: 93 - protein, 12 aa, Artificial Sequence
  Chimeric
CCQRDVELVFGS

SEQ ID NO: 94 - protein, 12 aa, Artificial Sequence
  Chimeric
CCRADFEVGNGG

SEQ ID NO: 95 - protein, 12 aa, Artificial Sequence
  Chimeric
CCVSDEPAGVRD

SEQ ID NO: 96 - protein, 12 aa, Artificial Sequence
  Chimeric
GAGWQEKDKELR

SEQ ID NO: 97 - protein, 12 aa, Artificial Sequence
  Chimeric
GAMTAGQLSDLP

SEQ ID NO: 98 - protein, 12 aa, Artificial Sequence
  Chimeric
VAGGQVVDRELK

SEQ ID NO: 99 - protein, 12 aa, Artificial Sequence
  Chimeric
KAGEQAMDMELR

SEQ ID NO: 100 - protein, 11 aa, Artificial Sequence
  Chimeric
RGRNQIMDLEI

SEQ ID NO: 101 - protein, 11 aa, Artificial Sequence
  Chimeric
QIDRQITDTLL

SEQ ID NO: 102 - protein, 11 aa, Artificial Sequence
  Chimeric
REQQISDVPRV

SEQ ID NO: 103 - protein, 12 aa, Artificial Sequence
  Chimeric
CQAMDAEILNQV

SEQ ID NO: 104 - protein, 11 aa, Artificial Sequence
  Chimeric
GQMMDTELLNR

SEQ ID NO: 105 - protein, 11 aa, Artificial Sequence
  Chimeric
SMEGQVRDIQV

SEQ ID NO: 106 - protein, 11 aa, Artificial Sequence
  Chimeric
YQQRDLELLAE

SEQ ID NO: 107 - protein, 11 aa, Artificial Sequence
  Chimeric
SMGQKVDRELV

SEQ ID NO: 108 - protein, 11 aa, Artificial Sequence
  Chimeric
SMGQEVDRELV

SEQ ID NO: 109 - protein, 11 aa, Artificial Sequence
  Chimeric
AENDQMVDWEI

SEQ ID NO: 110 - protein, 11 aa, Artificial Sequence
  Chimeric
GGWQESDIPGR

SEQ ID NO: 111 - protein, 11 aa, Artificial Sequence
  Chimeric
GGWQEKDKELR

SEQ ID NO: 112 - protein, 12 aa, Artificial Sequence
  Chimeric
HCCRIDREVSGA

SEQ ID NO: 113 - protein, 12 aa, Artificial Sequence
  Chimeric
CAPGMGCWESVK

SEQ ID NO: 114 - protein, 17 aa, Artificial Sequence
  Chimeric
SCREVWLGGSEMIMDCE

SEQ ID NO: 115 - protein, 17 aa, Artificial Sequence
  Chimeric
SCPAFPREGDLCAPPTV

SEQ ID NO: 116 - protein, 17 aa, Artificial Sequence
  Chimeric
FCPEPICSPPLSRMTLS

SEQ ID NO: 117 - protein, 17 aa, Artificial Sequence
  Chimeric
ECNQNLSGSLRHVDLNC

SEQ ID NO: 118 - protein, 17 aa, Artificial Sequence
  Chimeric
RCDQQLPRDSYTFCMMS

SEQ ID NO: 119 - protein, 17 aa, Artificial Sequence
  Chimeric
HCQQVFFPQDYLWCQRG

SEQ ID NO: 120 - protein, 17 aa, Artificial Sequence
  Chimeric
DCEEPMCSPVLLQKLKP

SEQ ID NO: 121 - protein, 17 aa, Artificial Sequence
  Chimeric
NCQDQMLREDAGCWSKI

SEQ ID NO: 122 - protein, 17 aa, Artificial Sequence
  Chimeric
HCEEPEYSPATRVFCGR

SEQ ID NO: 123 - protein, 17 aa, Artificial Sequence
  Chimeric
DCDWINPPDPPHFWKDT

SEQ ID NO: 124 - protein, 17 aa, Artificial Sequence
  Chimeric
ACFSRNGQVTDVPHSCY

SEQ ID NO: 125 - protein, 17 aa, Artificial Sequence
  Chimeric
KCPTYPKPNDRCLWPVP

SEQ ID NO: 126 - protein, 17 aa, Artificial Sequence
  Chimeric
YCPKYPLEGDCLLDNDY

SEQ ID NO: 127 - protein, 17 aa, Artificial Sequence
  Chimeric
RCEEWLCIPPAPAFAPP

SEQ ID NO: 128 - protein, 17 aa, Artificial Sequence
  Chimeric
TCGQSELRCASLETHHV

SEQ ID NO: 129 - protein, 16 aa, Artificial Sequence
  Chimeric
NCNDNPMLDCMPAWSS

SEQ ID NO: 130 - protein, 12 aa, Artificial Sequence
  Chimeric
DALDERAWRARA

SEQ ID NO: 131 - protein, 12 aa, Artificial Sequence
  Chimeric
SCQGREVRRECW

SEQ ID NO: 132 - protein, 12 aa, Artificial Sequence
  Chimeric
VCDECVSRELAL

SEQ ID NO: 133 - protein, 12 aa, Artificial Sequence
  Chimeric
WCLEPECAPGLL

SEQ ID NO: 134 - protein, 12 aa, Artificial Sequence
  Chimeric
DCLSKGQMADLC

SEQ ID NO: 135 - protein, 12 aa, Artificial Sequence
  Chimeric
VCDECVSRELAL

SEQ ID NO: 136 - protein, 12 aa, Artificial Sequence
  Chimeric
GCPTWPRVGDHC

SEQ ID NO: 137 - protein, 12 aa, Artificial Sequence
  Chimeric
RCQSARVVPECW

SEQ ID NO: 138 - protein, 12 aa, Artificial Sequence
  Chimeric
SCAPSGDCGYKG

SEQ ID NO: 139 - protein, 12 aa, Artificial Sequence
  Chimeric
GCPMWPQPDDEC

SEQ ID NO: 140 - protein, 12 aa, Artificial Sequence
  Chimeric
ECPRWPLMGDGC

SEQ ID NO: 141 - protein, 12 aa, Artificial Sequence
  Chimeric
GCQVGELVWCRE

SEQ ID NO: 142 - protein, 12 aa, Artificial Sequence
  Chimeric
QCVRDGTRKVCM

SEQ ID NO: 143 - protein, 12 aa, Artificial Sequence
  Chimeric
TCLVDRQESDVC

SEQ ID NO: 144 - protein, 12 aa, Artificial Sequence
  Chimeric
DCVVDGDRLVCL

SEQ ID NO: 145 - protein, 12 aa, Artificial Sequence
  Chimeric
RCEQGALRCVGE

SEQ ID NO: 146 - protein, 12 aa, Artificial Sequence
  Chimeric
VCPPGWKNLGCN

SEQ ID NO: 147 - protein, 12 aa, Artificial Sequence
  Chimeric
MCQGWEIVSECW

SEQ ID NO: 148 - protein, 25 aa, Artificial Sequence
  Chimeric
ADGAGCFMNKQMADLELCPREAAEA

SEQ ID NO: 149 - protein, 25 aa, Artificial Sequence
  Chimeric
ADGAGCFMNKQMADLELCPRTAAEA

SEQ ID NO: 150 - protein, 25 aa, Artificial Sequence
  Chimeric
ADGAACFMNKQMADLELCPRVAAEA

SEQ ID NO: 151 - protein, 25 aa, Artificial Sequence
  Chimeric
ADGAGCFINKQLADLELCPRVAAEA

SEQ ID NO: 152 - protein, 25 aa, Artificial Sequence
  Chimeric
ADGAGCFINKQLADLELCPREAAEA

SEQ ID NO: 153 - protein, 25 aa, Artificial Sequence
  Chimeric
ADGAGCFMNKQLADLEMCPRDDAEA

SEQ ID NO: 154 - protein, 25 aa, Artificial Sequence
  Chimeric
ADGAGCFMNKQLADPELCPREAEEA

SEQ ID NO: 155 - protein, 25 aa, Artificial Sequence
  Chimeric
ADGAGCFMNKQLVDLELCPRGAAEA

SEQ ID NO: 156 - protein, 25 aa, Artificial Sequence
  Chimeric
ADGAGCFMNNQLADWELCPRAAAEA

SEQ ID NO: 157 - protein, 25 aa, Artificial Sequence
  Chimeric
ADGAGCFMNKQMADWEMCPRAAAEA

SEQ ID NO: 158 - protein, 25 aa, Artificial Sequence
  Chimeric
ADGAGCFMNKQQADLELCPRGAAEA

SEQ ID NO: 159 - protein, 25 aa, Artificial Sequence
  Chimeric
ADGAECFMNKQLADSELCPRVAAEA

SEQ ID NO: 160 - protein, 25 aa, Artificial Sequence
  Chimeric
ADGAGCFMNKQLADLELCPREAAEA

SEQ ID NO: 161 - protein, 25 aa, Artificial Sequence
  Chimeric
ADGAGCFINMQMADQELCPRAAAEA

SEQ ID NO: 162 - protein, 25 aa, Artificial Sequence
  Chimeric
ADGAGCFINKQMSDFELCPREAGEA

SEQ ID NO: 163 - protein, 25 aa, Artificial Sequence
  Chimeric
ADGAGCFINKQMADLELCTREAAEA

SEQ ID NO: 164 - protein, 25 aa, Artificial Sequence
  Chimeric
ADGAGCFINKQMADLELCPRQAAEA

SEQ ID NO: 165 - protein, 25 aa, Artificial Sequence
  Chimeric
ADGAGCFINNQMADLELCPRGGAEA

SEQ ID NO: 166 - protein, 25 aa, Artificial Sequence
  Chimeric
ADGAGCFINKQMADWELCPREGAEA

SEQ ID NO: 167 - protein, 25 aa, Artificial Sequence
  Chimeric
ADGAGCFINKQMADLELCPSQAAEA

SEQ ID NO: 168 - protein, 25 aa, Artificial Sequence
  Chimeric
ADGAGCFINKQMADLELCPREGAEA

SEQ ID NO: 169 - protein, 25 aa, Artificial Sequence
  Chimeric
ADGAGCFINKQMADSELCPREPAEA

SEQ ID NO: 170 - protein, 25 aa, Artificial Sequence
  Chimeric
ADGAGCFIKKQMADLELCPREAWEA

SEQ ID NO: 171 - protein, 25 aa, Artificial Sequence
  Chimeric
ADGAECFINKQMADRELCAREVAEA

SEQ ID NO: 172 - protein, 25 aa, Artificial Sequence
  Chimeric
ADGAGCFIDKQMADLELCPRAAAEA

SEQ ID NO: 173 - protein, 25 aa, Artificial Sequence
  Chimeric
ADGAGCFINKQMADLELCRREAGEA

SEQ ID NO: 174 - protein, 25 aa, Artificial Sequence
  Chimeric
ADGAGCFKNKQMVDSELCARQAAEA

SEQ ID NO: 175 - protein, 25 aa, Artificial Sequence
  Chimeric
ADGAGCFQNKQMADLELCPREAAEA

SEQ ID NO: 176 - protein, 25 aa, Artificial Sequence
  Chimeric
ADGAECFINKQRADLELCPGEAAEA

SEQ ID NO: 177 - protein, 25 aa, Artificial Sequence
  Chimeric
ADGAGCFINKQMADSELCPAAAAEA

SEQ ID NO: 178 - protein, 25 aa, Artificial Sequence
  Chimeric
ADGAGCFINRQMADPELCPREAAEA

SEQ ID NO: 179 - protein, 25 aa, Artificial Sequence
  Chimeric
ADGAGCFIEKQMADMELCQARAAEA

SEQ ID NO: 180 - protein, 25 aa, Artificial Sequence
  Chimeric
ADGAGCFINKQMADWELCPREAAEA

SEQ ID NO: 181 - protein, 25 aa, Artificial Sequence
  Chimeric
ADGAGCFINKQMADWELCPREAAEA

SEQ ID NO: 182 - protein, 25 aa, Artificial Sequence
  Chimeric
ADGAGCFIEKQMADMELCQRETAEA

SEQ ID NO: 183 - protein, 25 aa, Artificial Sequence
  Chimeric
ADGAGCFINKQMADMELCPREAAEA

SEQ ID NO: 184 - protein, 25 aa, Artificial Sequence
  Chimeric
ADGAGCFINKQMADLELCPREAAEA

SEQ ID NO: 185 - protein, 25 aa, Artificial Sequence
  Chimeric
ADGAGCFRNKQMADLELCPREAAEA

SEQ ID NO: 186 - protein, 25 aa, Artificial Sequence
  Chimeric
ADGAGCFINKQMADLELCPARAAEA

SEQ ID NO: 187 - protein, 25 aa, Ala Asp Gly Ala Gly Cys Phe Ile Asn Arg Gln Leu u
  Chimeric
ADGAGCFINRQLADMELCSRGAAEA

SEQ ID NO: 188 - protein, 25 aa, Artificial Sequence
  Chimeric
ADGAECFINRQMADLELCGREAAEA

SEQ ID NO: 189 - protein, 25 aa, Artificial Sequence
  Chimeric
ADGAGCFISPQLADWKRCMREAAEA

SEQ ID NO: 190 - protein, 25 aa, Artificial Sequence
  Chimeric
ADGAGCSIHTQMADWERCLREGAEA

SEQ ID NO: 191 - protein, 25 aa, Artificial Sequence
  Chimeric
ADGAGCSIHRQMADWERCLREGAEA

SEQ ID NO: 192 - protein, 16 aa, Artificial Sequence
  Chimeric
CSSCDGGGHKPPTIQC

SEQ ID NO: 193 - protein, 20 aa, Artificial Sequence
  Chimeric
CLQSSCDGGGHFPPTIQLLC